SEQUENCE LISTING (93 sequences)

SEQ ID NO: 1 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatggattaataggtctggtaagcagacatcgtacgcagactccgtgaagggccggttcaccatctccagagacaattccaagaacacgctgtatctgcaaatgaacagcctgagagccgaggacacggccgtatattactgtgcgaaacggagtaaggcgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcaggcatccaatttgcaaagtggggtcccatcaaggttcagtggcagtggatctgggacagatttcactctcaccatcagcagtctgcaacctgaagattttgcaacttactactgtcaacagatgcagagggcgcctagtacgttcggccaagggaccaaggtggaaatcaaacgggcggccgcacatcatcatcaccatcacggggccgcagaacaaaaactcatctcagaagaggatctgaattag

SEQ ID NO: 2 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSWINRSGKQTSYADSVKGRFTISRDNSKNTLYLQMNSLRAEDTAVYYCAKRSKAFDYWGQGTLVTVSSGGGGSGGGGSGGGGSTDIQMTQSPSSLSASVGDRVTITCRASQSISSYLNWYQQKPGKAPKLLIYQASNLQSGVPSRFSGSGSGTDFTLTISSLQPEDFATYYCQQMQRAPSTFGQGTKVEIKRAAAHHHHHHGAAEQKLISEEDLN

SEQ ID NO: 3 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagggatttctcagcgtggtactcatacaacttacgcagactccgtgaagggccggttcaccatctccagagacaattccaagaacacgctgtatctgcaaatgaacagcctgagagccgaggacacggccgtatattactgtgcgaaaggtcggcggcggtttgactactggggccagggaaccctggtcaccgtctcgagcggtggaggcggttcaggcggaggtggcagcggcggtggcgggtcgacggacatcaagatgacccagtctccatcctccctgtctgcatctgtaggagacagagtcaccatcacttgccgggcaagtcagagcattagcagctatttaaattggtatcagcagaaaccagggaaagcccctaagctcctgatctatgatgcatccactttgcaaagtggggtcccatcaaggttcagtggcagtggatctgggacagatttcactctcaccatcagcagtctgcaacctgaagattttgcaacttactactgtcaacagacgcagcctacgcctcatacgttcggccaagggaccaaggtggaaatcaaacgggcggccgcacatcatcatcaccatcacggggccgcagaacaaaaactcatctcagaagaggatctgaattag

SEQ ID NO: 4 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSGISQRGTHTTYADSVKGRFTISRDNSKNTLYLQMNSLRAEDTAVYYCAKGRRRFDYWGQGTLVTVSSGGGGSGGGGSGGGGSTDIKMTQSPSSLSASVGDRVTITCRASQSISSYLNWYQQKPGKAPKLLIYDASTLQSGVPSRFSGSGSGTDFTLTISSLQPEDFATYYCQQTQPTPHTFGQGTKVEIKRAAAHHHHHHGAAEQKLISEEDLN

SEQ ID NO: 5 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattccttctaagggtcatacgacacagtacgcagactccgtgaagggccggttcaccatctccagagacaattccaagaacacgctgtatctgcaaatgaacagcctgagagccgaggacacggccgtatattactgtgcgaaaaagagggggacgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcgtgcatcccatttgcaaagtggggtcccatcaaggttcagtggcagtggatctgggacagatttcactctcaccatcagcagtctgcaacctgaagattttgcaacttactactgtcaacagaatcgtaagtatcctagtacgttcggccaagggaccaaggtggaaatcaaacgggcggccgcacatcatcatcaccatcacggggccgcagaacaaaaactcatctcagaagaggatctgaattag

SEQ ID NO: 6 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSSIPSKGHTTQYADSVKGRFTISRDNSKNTLYLQMNSLRAEDTAVYYCAKKRGTFDYWGQGTLVTVSSGGGGSGGGGSGGGGSTDIQMTQSPSSLSASVGDRVTITCRASQSISSYLNWYQQKPGKAPKLLIYRASHLQSGVPSRFSGSGSGTDFTLTISSLQPEDFATYYCQQNRKYPSTFGQGTKVEIKRAAAHHHHHHGAAEQKLISEEDLN

SEQ ID NO: 7 - DNA, 654 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcacagattcggaattctggtcagtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagtgcatccaggttgcaaagtggggtcccatcaaggttcagtggcagtggatctgggacagatttcactctcaccatcagcagtctgcaacctgaagattttgcaacttactactgtcaacagatgcagactgctcctaatacgttcggccaagggaccaaggtggaaatcaaacgggcggccgcacatcatcatcaccatcacggggccgcagaacaaaaactcatctcagaagaggatctgaatggggccgcatag

SEQ ID NO: 8 - protein, 217 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSQIRNSGQSTDIQMTQSPSSLSASVGDRVTITCRASQSISSYLNWYQQKPGKAPKLLIYSASRLQSGVPSRFSGSGSGTDFTLTISSLQPEDFATYYCQQMQTAPNTFGQGTKVEIKRAAAHHHHHHGAAEQKLISEEDLNGAA

SEQ ID NO: 9 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaattattgatccgctgggtcaggctacaaagtacgcagactccgtgaagggcaggttcaccatctccagagacaattccaagaacacgctgtatctgcaaatgaacagcctgagagccgaggacacggccgtatattactgtgcgaaaaggcaatcgacgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcgggcatcctatttgcaaagtggggtcccatcaaggttcagtggcagtggatctgggacagatttcactctcaccatcagcagtctgcaacctgaagattttgcaacttactactgtcaacagaagcagaatccgnctcctacgttcggccaagggaccaaggtggaaatcaaacgggcggccgcccatcatcatcaccatcacggggccgcagaacaaaaactcatctcagaagaggatctgaattag

SEQ ID NO: 10 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSIIDPLGQATKYADSVKGRFTISRDNSKNTLYLQMNSLRAEDTAVYYCAKRQSTFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQKQNPXPTFGQGTKVEIKRAAAHHHHHHGAAEQKLISEEDLN

SEQ ID NO: 11 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagcgatttcgaatggtggtgtgcagacagcgtacgcagactccgtgaagggccggttcaccatctccagagacaattccaagaacacgctgtatctgcaaatgaacagcctgagagccgaggacacggccgtatattactgtgcgaaaaatactggttcgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgcggcatcccgtttgcaaagtggggtcccatcaaggttcagtggcagtggatctgggacagatttcactctcaccatcagcagtctgcaacctgaagattttgcaacttactactgtcaacagtcgcaggcgaggcctgtgacgttcggccaagggaccaaggtggaaatcaaacgggcggccgcccatcatcatcaccatcacggggccgcagaacaaaaactcatctcagaagaggatctgaattag

SEQ ID NO: 12 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSAISNGGVQTAYADSVKGRFTISRDNSKNTLYLQMNSLRAEDTAVYYCAKNTGSFDYWGQGTLVTVSSGGGGSGGGGSGGGGSTDIQMTQSPSSLSASVGDRVTITCRASQSISSYLNWYQQKPGKAPKLLIYAASRLQSGVPSRFSGSGSGTDFTLTISSLQPEDFATYYCQQSQARPVTFGQGTKVEIKRAAAHHHHHHGAAEQKLISEEDLN

SEQ ID NO: 13 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattcagaagtcgggtgagaagacacattacgcagactccgtgaagggccggttcaccatctncagagacaattccaagaacacgctgtatctgcaaatgaacagcctgagagccgaggacacggncgtatattactgtgcgaaaaagaggacgctgtttgactactggggccagggaaccctggtcaccgtctcgagcggtggaggcggttcaggcngaggtggcagctgcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctataaggcatcccatttgcaaagtggggtcccatcaaggttcagtggcagtggatctgggacagatttcactctcaccatcagcagtctgcaacctgaagattttgcaacttactactgtcaacaggctaggtctcggcctacgacgttcggccaagggaccaaggtggaaatcaaacgggcggccgcacatcatcatcaccatcacggggccgcagaacaaaaactcatctcagaagaggatctgaattag

SEQ ID NO: 14 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSSIQKSGEKTHYADSVKGRFTIXRDNSKNTLYLQMNSLRAEDTXVYYCAKKRTLFDYWGQGTLVTVSSGGGGSGXGGSCGGGSTDIQMTQSPSSLSASVGDRVTITCRASQSISSYLNWYQQKPGKAPKLLIYKASHLQSGVPSRFSGSGSGTDFTLTISSLQPEDFATYYCQQARSRPTTFGQGTKVEIKRAAAHHHHHHGAAEQKLISEEDLN

SEQ ID NO: 15 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaaatattaagcctactggtgcgcatacatagtacgcagactccgtgaagggccggttcaccatctccagagacaattccaagaacacgctgtatctgcaaatgaacagcctgagagccgaggacacggccgtatattactgtgcgaaaggtaagcgtacgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcaggcatccaatttgcaaagtggggtcccatcaaggttcagtggcagtggatctgggacagatttcactctcaccatcagcagtctgcaacctgaagattttgcaacttactactgtcaacagatgcagagggcgcctagtacgttcggccaagggaccaaggtggaaatcaaacgggcggccgcacatcatcatcaccatcacggggccgcagaacaaaaactcatctcagaagaggatctgaattag

SEQ ID NO: 16 - protein, 287 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSNIKPTGAHTYADSVKGRFTISRDNSKNTLYLQMNSLRAEDTAVYYCAKGKRTFDYWGQGTLVTVSSGGGGSGGGGSGGGGSTDIQMTQSPSSLSASVGDRVTITCRASQSISSYLNWYQQKPGKAPKLLIYQASNLQSGVPSRFSGSGSGTDFTLTISSLQPEDFATYYCQQMQRAPSTFGQGTKVEIKRAAAHHHHHHGAAEQKLISEEDLN

SEQ ID NO: 17 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaactattaagcgttcgggtacgtttacacagtacgcagactccgtgaagggccggttcaccatctccagagacaattccaagaacacgctgtatctgcaaatgaacagcctgagagccgaggacacggccgtatattactgtgcgaaaggtaatcattcttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatcgtgcatcccgtttgcaaagtggggtcccatcaaggttcagtggcagtggatctgggacagatttcactctcaccatcagcagtctgcaacctgaagattttgcaacttactactgtcaacaggataagaagcctcctgtgacgttcggccaagggaccaaggtggaaatcaaacgggcggccgcacatcatcatcaccatcacggggccgcagaacaaaaactcatctcagaagaggatctgaattag

SEQ ID NO: 18 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSTIKRSGTFTQYADSVKGRFTISRDNSKNTLYLQMNSLRAEDTAVYYCAKGNHSFDYWGQGTLVTVSSGGGGSGGGGSGGGGSTDIQMTQSPSSLSASVGDRVTITCRASQSISSYLNWYQQKPGKAPKLLIYRASRLQSGVPSRFSGSGSGTDFTLTISSLQPEDFATYYCQQDKKPPVTFGQGTKVEIKRAAAHHHHHHGAAEQKLISEEDLN

SEQ ID NO: 19 - protein, 22 aa, Bacillus subtilis
  Single chain antibody
MLEGKVKWFNSEKGFGFIEVEG

SEQ ID NO: 20 - protein, 35 aa, Bacillus subtilis
MLEGKVKWFNSEKGFGFIEVEGQDDVFVHFSAIQG

SEQ ID NO: 21 - protein, 6 aa, Adenovirus type 2
GAITIG

SEQ ID NO: 22 - protein, 8 aa, Adenovirus type 2
NSGAITIG

SEQ ID NO: 23 - protein, 12 aa, Adenovirus type 2
LSFDNSGAITIG

SEQ ID NO: 24 - protein, 25 aa, Adenovirus type 2
AMITKLGSGLSFDNSGAITIGNKND

SEQ ID NO: 25 - protein, 40 aa, Adenovirus type 2
IKTKIGSGIDYNENGAMITKLGSGLSFDNSGAITIGNKND

SEQ ID NO: 26 - protein, 51 aa, Antheraea polyphemus
SYGGEGIGNVAVAGELPVAGKTAVAGRVPIIGAVGFGGPAGAAGAVSIAGR

SEQ ID NO: 27 - protein, 18 aa, Bombyx mori
  VARIANT 10: Xaa = Gly or Asp
GNLPFLGTAXVAGEFPTA

SEQ ID NO: 28 - protein, 45 aa, Bombyx mori
FREIKGYEYQLYVYASDKLFRADISEDYKTRGRKLLRFNGPVPPP

SEQ ID NO: 29 - protein, 50 aa, Dioscoreophyllum cumminsii
GEWEIIDIGPFTQNLGKFAVDEENKIGQYGRLTFNKVIRPCMKKTIYEEN

SEQ ID NO: 30 - protein, 150 aa, Escherichia coli
MKLLKVAAIAAIVFSGSALAGVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAADQTASNSSVNVTQVGFGNNATAHQY

SEQ ID NO: 31 - protein, 152 aa, Salmonella typhimurium
MKLLKVAAFAAIVVSGSAVAGVVPQWGGGGNHNGGGNSSGPDSTLSIYQYGSANAALALQSDARKSETTITQSGYGNGADVGQGADNSTIELTQNGFRNNATIDQWNAKNSDITVGQYGGNNAALVNQTASDSSVMVRQVGFGNNAPANQYN

SEQ ID NO: 32 - protein, 685 aa, Saccharomyces cerevisiae
MSDSNQGNNQQNYQQYSQNGNQQQGNNRYQGYQAYNAQAQPAGGYYQNYQGYSGYQQGGYQQYNPDAGYQQQYNPQGGYQQYNPQGGYQQQFNPQGGRGNYKNFNYNNNLQGYQAGFQPQSQGMSLNDFQKQQKQAAPKPKKTLKLVSSSGIKLANATKKVGTKPAESDKKEEEKSAETKEPTKEPTKVEEPVKKEEKPVQTEEKTEEKSELPKVEDLKISESTHNTNNANVTSADALIKEQEEEVDDEVVNDMFGGKDHVSLIFMGHVDAGKSTMGGNLLYLTGSVDKRTIEKYEREAKDAGRQGWYLSWVMDTNKEERNDGKTIEVGKAYFETEKRRYTILDAPGHKMYVSEMIGGASQADVGVLVISARKGEYETGFERGGQTREHALLAKTQGVNKMVVVVNKMDDPTVNWSKERYDQCVSNVSNFLRAIGYNIKTDVVFMPVSGYSGANLKDHVDPKECPWYTGPTLLEYLDTMNHVDRHINAPFMLPIAAKMKDLGTIVEGKIESGHIKKGQSTLLMPNKTAVEIQNIYNETENEVDMAMCGEQVKLRIKGVEEEDISPGFVLTSPKNPIKSVTKFVAQIAIVELKSIIAAGFSCVMHVHTAIEEVHIVKLLHKLEKGTNRKSKKPPAFAKKGMKVIAVLETEAPVCVETYQDYPQLGRFTLRDQGTTIAIGKIVKIAE

SEQ ID NO: 33 - protein, 33 aa, Myoxocephalus scorpius
MNAPARAAAKTAADALAAAKKTAADAAAAAAAA

SEQ ID NO: 34 - protein, 7 aa, Podospora anserine
GNNQQNY

SEQ ID NO: 35 - protein, 6 aa, Artificial Sequence
  Adenovirus fragment
GAITIG

SEQ ID NO: 36 - protein, 8 aa, Artificial Sequence
  Adenovirus fragment
NSGAITIG

SEQ ID NO: 37 - protein, 12 aa, Artificial Sequence
  Adenovirus fragment
LSFDNSGAITIG

SEQ ID NO: 38 - protein, 25 aa, Artificial Sequence
  Adenovirus fragment
AMITKLGSGLSFDNSGAITIGNKND

SEQ ID NO: 39 - protein, 41 aa, Artificial Sequence
  Adenovirus fragment
PIKTKIGSGIDYNENGAMITKLGSGLSFDNSGAITIGNKND

SEQ ID NO: 40 - protein, 22 aa, Bacillus subtilis
MLEGKVKWFNSEKGFGFIEVEG

SEQ ID NO: 41 - protein, 35 aa, Bacillus subtilis; identical to SEQ ID NO: 20

SEQ ID NO: 42 - protein, 51 aa, Bombyx mori; identical to SEQ ID NO: 26

SEQ ID NO: 43 - protein, 18 aa, Bombyx mori
GNLPFLGTAGVAGEFPTA

SEQ ID NO: 44 - protein, 17 aa, Artificial Sequence
  Synthetic
SIRELEARIRELELRIG

SEQ ID NO: 45 - protein, 9 aa, Human papillomavirus
RAHYNIVTF

SEQ ID NO: 46 - protein, 7 aa, Saccharomyces cerevisiae
GNNQQNY

SEQ ID NO: 47 - protein, 6 aa, Saccharomyces cerevisiae
NNQQNY

SEQ ID NO: 48 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagggatttctcagcgtggtactcatacaacttacgcagactccgtgaagggccggttcaccatctccagagacaattccaagaacacgctgtatctgcaaatgaacagcctgagagccgaggacacggccgtatattactgtgcgaaaggtcggcggcggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 49 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSGISQRGTHTTYADSVKGRFTISRDNSKNTLYLQMNSLRAEDTAVYYCAKGRRRFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 50 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattaagtatagtggtcaggcnacaacttacgcagactccgtgaagggccggttcaccatctccagagacaattccaagaacacgctgtatctgcaaatgaacagcctgagagccgaggacacggccgtatattactgtgcgaaaaatcatcagctttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 51 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSSIKYSGQATTYADSVKGRFTISRDNSKNTLYLQMNSLRAEDTAVYYCAKNHQLFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 52 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaactattaagcgttcgggtacgtttacacagtacgcagactccgtgaagggccggttcaccatctccagagacaattccaagaacacgctgtatctgcaaatgaacagcctgagagccgaggacacggccgtatattactgtgcgaaaggtaatcattcttttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 53 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSTIKRSGTFTQYADSVKGRFTISRDNSKNTLYLQMNSLRAEDTAVYYCAKGNHSFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 54 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatggattaataggtctggtaagcagacatcgtacgcagactccgtgaagggccggttcaccatctccagagacaattccaagaacacgctgtatctgcaaatgaacagcctgagagccgaggacacggccgtatattactgtgcgaaacggagtaaggcgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 55 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSWINRSGKQTSYADSVKGRFTISRDNSKNTLYLQMNSLRAEDTAVYYCAKRSKAFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 56 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcaattattgatccgctgggtcaggctacaaagtacgcagactccgtgaagggcaggttcaccatctccagagacaattccaagaacacgctgtatctgcaaatgaacagcctgagagccgaggacacggccgtatattactgtgcgaaaaggcaatcgacgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 57 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSIIDPLGQATKYADSVKGRFTISRDNSKNTLYLQMNSLRAEDTAVYYCAKRQSTFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 58 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcagcgatttcgaatggtggtgtgcagacagcgtacgcagactccgtgaagggccggttcaccatctccagagacaattccaagaacacgctgtatctgcaaatgaacagcctgagagccgaggacacggccgtatattactgtgcgaaaaatactggttcgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 59 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSAISNGGVQTAYADSVKGRFTISRDNSKNTLYLQMNSLRAEDTAVYYCAKNTGSFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 60 - protein, 11 aa, Artificial Sequence
  Single chain antibody region
SIQKSGEKTHY

SEQ ID NO: 61 - protein, 4 aa, Artificial Sequence
  Single chain antibody region
KRTL

SEQ ID NO: 62 - protein, 6 aa, Artificial Sequence
  Single chain antibody region
ARSRPT

SEQ ID NO: 63 - protein, 11 aa, Artificial Sequence
  Single chain antibody region
GISQRGTHTTY

SEQ ID NO: 64 - protein, 4 aa, Artificial Sequence
  Single chain antibody region
GRRR

SEQ ID NO: 65 - protein, 6 aa, Artificial Sequence
  Single chain antibody region
TQPTPH

SEQ ID NO: 66 - protein, 11 aa, Artificial Sequence
  Single chain antibody region
SIKYSGQATTY

SEQ ID NO: 67 - protein, 4 aa, Artificial Sequence
  Single chain antibody region
GNKR

SEQ ID NO: 68 - protein, 6 aa, Artificial Sequence
  Single chain antibody region
AKYPPP

SEQ ID NO: 69 - protein, 11 aa, Artificial Sequence
  Single chain antibody region
TIKRSGTFTQY

SEQ ID NO: 70 - protein, 4 aa, Artificial Sequence
  Single chain antibody region
GNHS

SEQ ID NO: 71 - protein, 6 aa, Artificial Sequence
  Single chain antibody region
DKKPPV

SEQ ID NO: 72 - protein, 11 aa, Artificial Sequence
  Single chain antibody region
TISMSGKRTQY

SEQ ID NO: 73 - protein, 4 aa, Artificial Sequence
  Single chain antibody region
RSKA

SEQ ID NO: 74 - protein, 6 aa, Artificial Sequence
  Single chain antibody region
MQRAPS

SEQ ID NO: 75 - protein, 11 aa, Artificial Sequence
  Single chain antibody region
IIDPLGAQTKY

SEQ ID NO: 76 - protein, 4 aa, Artificial Sequence
  Single chain antibody region
RQST

SEQ ID NO: 77 - protein, 6 aa, Artificial Sequence
  Single chain antibody region
KQNPXP

SEQ ID NO: 78 - protein, 11 aa, Artificial Sequence
  Single chain antibody region
AISNGGVQTAY

SEQ ID NO: 79 - protein, 4 aa, Artificial Sequence
  Single chain antibody region
NTGS

SEQ ID NO: 80 - protein, 6 aa, Artificial Sequence
  Single chain antibody region
SQARPV

SEQ ID NO: 81 - DNA, 867 bp, Artificial Sequence
  Single chain antibody
atggagacagacacactcctgctatgggtactgctgctctgggttccaggttccactggtgacaagcttatggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattcagaagtcgggtgagaagacacattacgcagactccgtgaagggccggttcaccatctccagagacaattccaagaacacgctgtatctgcaaatgaacagcctgagagccgaggacacggccgtatattactgtgcgaaaaagaggacgctgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatagggcatcctacttgcaaagtggggtcccatcaaggttcagtggcagtggatctgggacagatttcactctcaccatcagcagtctgcaacctgaagattttgcaacttactactgtcaacagtcgtattcgctgcctactacgttcggccaagggaccaaggtggaaatcaaacgggcggccgcacatcatcatcaccatcacggggccgcagaacaaaaactcatctcagaagaggatctgaattag

SEQ ID NO: 82 - protein, 288 aa, Artificial Sequence
  Single chain antibody
METDTLLLWVLLLWVPGSTGDKLMAEVQLLESGGGLVQPGGSLRLSCAASGFTFSSYAMSWVRQAPGKGLEWVSSIQKSGEKTHYADSVKGRFTISRDNSKNTLYLQMNSLRAEDTAVYYCAKKRTLFDYWGQGTLVTVSSGGGGSGGGGSGGGGSTDIQMTQSPSSLSASVGDRVTITCRASQSISSYLNWYQQKPGKAPKLLIYRASYLQSGVPSRFSGSGSGTDFTLTISSLQPEDFATYYCQQSYSLPTTFGQGTKVEIKRAAAHHHHHHGAAEQKLISEEDLN

SEQ ID NO: 83 - protein, 15 aa, Artificial Sequence
  Synthetic
AALALPAVLLALLAP

SEQ ID NO: 84 - protein, 17 aa, Artificial Sequence
  Synthetic
KGEGAAVLLPVLLAAPG

SEQ ID NO: 85 - DNA, 804 bp, Artificial Sequence
  Single chain antibody
gaggtgcagctgttggagtctgggggaggcttggtacagcctggggggtccctgaggctctcctgtgcagcctctgggttcacctttagcagctatgccatgagctgggcccgccaggctccagggaaggggctggggtgggtctcatctattcagaagtcgggtgagaagacacattacgcagactccgtgaagggccggttcaccatctccagagacaattccaagaacacgctgtatctgcaaatgaacagcctgagagccgaggacacggccgtatattactgcgcgaaaaagaggacgctgtttgactactggggccagggaaccctggccaccgtcccgagtggtggaggcggttcaggcggaggtggcagcggcggtggcgggtcgacggacatccagacgacccagtccccgtcctccctgtctgcatctgtaggagacagagtcaccatcacttgccgggcaagtcagagcattagcagctatttaaattggtatcggcagaaaccagggaaagcccctaggctcctgatctataaggcaccccatttgcaaagtggggccccatcaaggctcagtggcagtggatctgggacagacttcactctcaccatcagcagtccgcaacctgaggattttgcaacttactactgtcagcgggctaggtctcggcctacgacgttcggccaagggaccaaggtggaaatcaaacgggcggccgctcgaggagggcccgaacaaaaactcatctcagaagaggatctgaatagcgccgtcgaccatcatcatcatcatcat

SEQ ID NO: 86 - protein, 268 aa, Artificial Sequence
  Single chain antibody
EVQLLESGGGLVQPGGSLRLSCAASGFTFSSYAMSWARQAPGKGLGWVSSIQKSGEKTHYADSVKGRFTISRDNSKNTLYLQMNSLRAEDTAVYYCAKKRTLFDYWGQGTLATVPSGGGGSGGGGSGGGGSTDIQTTQSPSSLSASVGDRVTITCRASQSISSYLNWYRQKPGKAPRLLIYKAPHLQSGAPSRLSGSGSGTDFTLTISSPQPEDFATYYCQRARSRPTTFGQGTKVEIKRAAARGGPEQKLISEEDLNSAVDHHHHHH

SEQ ID NO: 87 - DNA, 804 bp, Artificial Sequence
  Single chain antibody
gaggtgcagctgttggagcctgggggaggcttggtacagcctggggggtccctgaggccctcctgtgcagcctctgggttcacctttagctgctatgccatgagctgggcccgccaggctccagggaaggggctggggtgggtctcatctattcagaagtcgggtgaggagacacattacgcagactccgtgaagggccggttcaccatctccagagacaattccaagaacacgctgtatctgcaaatgaacagcctgggagccgaggacacggccgtatattactgcgcgaagaagaggacgctgtttgactactggggccagggaaccctggccaccgtcccgagtggtggaggcggttcaggtggaggtggcagcggcggtggcgggtcgacggacatccagacgacccagtccccgtcctccctgtcagcatctgtaggagacagagtcaccatcacttgccgggcacgtcagagcattagcagctatttaaattggtatcggcagaaaccagggaaagcccctaggctcctgatctataaggcaccccatttgcaaagtggggccccatcaaggctcagtggcagtggatctgggtcagacttcactctcaccatcagcagtccgcaacctgaggattttgcaacttactactgtcagcgggctaggtctcggcctacgacgttcggccaagggaccaangtggaaatcanacgggcggccgctcgaggagggcccgaacaaaaactcatctcagaagaggatctgaatagcgccgtcgaccatcatcatcatcatcat

SEQ ID NO: 88 - protein, 268 aa, Artificial Sequence
  Single chain antibody
EVQLLEPGGGLVQPGGSLRPSCAASGFTFSCYAMSWARQAPGKGLGWVSSIQKSGEETHYADSVKGRFTISRDNSKNTLYLQMNSLGAEDTAVYYCAKKRTLFDYWGQGTLATVPSGGGGSGGGGSGGGGSTDIQTTQSPSSLSASVGDRVTITCRARQSISSYLNWYRQKPGKAPRLLIYKAPHLQSGAPSRLSGSGSGSDFTLTISSPQPEDFATYYCQRARSRPTTFGQGTXVEIXRAAARGGPEQKLISEEDLNSAVDHHHHHH

SEQ ID NO: 89 - DNA, 804 bp, Artificial Sequence
  Single chain antibody
gaggtgcagctgttggagactgggggaggcttggtacagcctggggggtccctgaggctctcctgtgcagcctccgggttcaccattagcagctatgccatgagctgggcccgccaggctccagggaaggggctggggtggttctcatctattcagaagtcgggtgagaagacacattacgcagactccgtgaagggccggttcaccatctccagagacaattccaagaacacgctgtatctgcaaatgaacagcctgagatccgaggacacggccgtatattactgcgcgaaaaagaggacgctgtttgactactggggccggggaaccctggccaccgtcccgagtggtggaggcggttctggcggaggtggcagcggcggtggcgggtcgtcggacatccagacgacccagtccccgtcctccctgtttgcatctgtaggagacggagtcaccatcacttgccgggcaagacagagcattagcagctatttaaattggtatcggcagaaaccagggaaagcccctaggctcctgatctataaggcaccccatttgcaaagtggggccccgtcaaggctcggtggcagtggatctgggacagacttcactctcaccatcagcagtccgcaacctgagggttctgcaacttactactgtcagcgggctaggtcttggcctacgacgttcggccaagggaccaaggtggaaatcaaacgggcggccgctcgaggagggcccgaacaaaaactcatctcagaagaggatctgaatagcgccgtcgaccatcatcatcatcatcat

SEQ ID NO: 90 - protein, 268 aa, Artificial Sequence
  Single chain antibody
EVQLLETGGGLVQPGGSLRLSCAASGFTISSYAMSWARQAPGKGLGWFSSIQKSGEKTHYADSVKGRFTISRDNSKNTLYLQMNSLRSEDTAVYYCAKKRTLFDYWGRGTLATVPSGGGGSGGGGSGGGGSSDIQTTQSPSSLFASVGDGVTITCRARQSISSYLNWYRQKPGKAPRLLIYKAPHLQSGAPSRLGGSGSGTDFTLTISSPQPEGSATYYCQRARSWPTTFGQGTKVEIKRAAARGGPEQKLISEEDLNSAVDHHHHHH

SEQ ID NO: 91 - DNA, 804 bp, Artificial Sequence
  Single chain antibody
gaggtgcagctgttggagtctgggggaggcttggtacagcctggggggtccccgaggctctcctgtgcagcctctgggttcacctttagcagctatgccatgagctgggcccgccagactccagggaaggggctggggtgggtctcatctattcagaagtcgggtgagaagacacattacgcagactccgtgaagggccggttcaccatctccagagacaattccaagaacacgctgtatctgcaagtgaacagcctgagagccggggacacggccgtatattactgcgcgaaaaagaggacgctgtttgactactggggccagggaaccctggccaccgtcccgagtggtgtaggtggttcaggcggaggtggcagcggcggtggcgggtcgacggacatccagacgacccagtccccgtcctccctgtctgcatctgtaggagacagagtctccaccacttgccgggcaagtcagagcattagcagctatttaaattggtatcggcagaaaccagggaaagccactaggctcctgatctataaggcaccccattcgcaaagtagggccccatcaaggctcagtggcagtggatctgggacagacttcactctcaccatcagcagtccgcancctgaggatcttgcaacttactactgtcagcgggctaggtctcggcctacgacgttcagccaggggaccaaggtggaaatcaaacgggcggccgctcgaggagggcccgaacaaaaactcatctcagaagaggatctgaatagcgccgtcgaccatcatcatcatcatcat

SEQ ID NO: 92 - protein, 268 aa, Artificial Sequence
  Single chain antibody
EVQLLESGGGLVQPGGSPRLSCAASGFTFSSYAMSWARQTPGKGLGWVSSIQKSGEKTHYADSVKGRFTISRDNSKNTLYLQVNSLRAGDTAVYYCAKKRTLFDYWGQGTLATVPSGVGGSGGGGSGGGGSTDIQTTQSPSSLSASVGDRVSTTCRASQSISSYLNWYRQKPGKATRLLIYKAPHSQSRAPSRLSGSGSGTDFTLTISSPXPEDLATYYCQRARSRPTTFSQGTKVEIKRAAARGGPEQKLISEEDLNSAVDHHHHHH

SEQ ID NO: 93 - protein, 147 aa, Gallus gallus
MRSLLILVLCFLPLAALGKVFGRCELAAAMKRHGLDNYRGYSLGNWVCVAKFESNFNTQATNRNTDGSTDYGILQINSRWWCNDGRTPGSRNLCNIPCSALLSSDITASVNCAKKIVSDGNGMSAWVAWRNRCKGTDVQAWIRGCRL

(19) United States
(12) Patent Application Publication
(10) Pub. No.: US 2010/0143365 A1
(43) Pub. Date: Jun. 10, 2010

(54) METHODS AND MATERIALS RELATED TO ANTI-AMYLOID ANTIBODIES

(75) Inventors: Todd E. Golde, Ponte Vedra Beach, FL (US); Yona R. Levites, Jacksonville, FL (US); Karen R. Jansen-West, Jacksonville Beach, FL (US); Pritam Das, Ponte Vedra Beach, FL (US); Becky Stodola, Verona, WI (US)

Correspondence Address: FISH & RICHARDSON P.C., PO BOX 1022, MINNEAPOLIS, MN, 55440-1022, US

(73) Assignee: MAYO FOUNDATION FOR MEDICAL EDUCATION AND RESEARCH, Rochester, MN (US)

(21) Appl. No.: 12/479,446

(22) Filed: Jun. 5, 2009

Related U.S. Application Data
(63) Continuation-in-part of application No. PCT/US2007/086843, filed on Dec. 7, 2007.
(60) Provisional application No. 60/869,064, filed on Dec. 7, 2006.

(51) Int. Cl. A61K 39/395; C07K 16/18

(52) U.S. Cl. 424/141.1; 530/387.1; 530/388.1; 424/130.1

(57) ABSTRACT

This document provides methods and materials related to anti-amyloid antibodies. For example, anti-amyloid antibodies, methods for making anti-amyloid antibodies, and methods for using an anti-amyloid antibody to treat or prevent an amyloid condition (e.g., AD) are provided.

### CROSS-REFERENCE TO RELATED APPLICATIONS

This application is a continuation-in-part application of International PCT Application Serial No. PCT/US2007/086843, filed Dec. 7, 2007, which claims the benefit of U.S. Provisional Application Ser. No. 60/869,064, filed Dec. 7, 2006, each of which are hereby incorporated by reference in their entirety.

### STATEMENT AS TO FEDERALLY SPONSORED RESEARCH

Funding for the work described herein was provided by the federal government under grant number AG 021875 awarded by the National Institute of Health. The federal government has certain rights in the invention.

### BACKGROUND

1. Technical Field

This document provides methods and materials related to anti-amyloid antibodies (e.g., anti-amyloid single-chain variable fragment (scFv) antibodies) and treating conditions associated with deposition of proteins as amyloid (e.g., Alzheimer's disease).

2. Background Information

Alzheimer's disease (AD) is the most common amyloidosis. In AD, the amyloid β protein (Aβ) accumulates as amyloid. It is generally acknowledged that the process that results in accumulation of Aβ as amyloid triggers the complex pathological changes that ultimately lead to cognitive dysfunction in Alzheimer's disease (AD). Aβ accumulates as amyloid in senile plaques and cerebral vessels, but it is also found in diffuse plaques recognized by antibodies but not classic amyloid stains. Although a minor component of the Aβ species produced by processing of amyloid precursor protein (APP), the highly amyloidogenic 42 amino acid form of Aβ (Aβ1-42) and amino terminally truncated forms of Aβ1-42 (Aβx-42) are the predominant species of Aβ typically found in both diffuse and senile plaques within the AD brain. However, many other forms of Aβ (e.g., Aβ1-40 or Aβx-40) are also present, especially in cerebrovascular amyloid deposits. In any case, Aβ in its non-aggregated form is not harmful, but can be when it aggregates into amyloid.

### SUMMARY

This document provides methods and materials related to anti-amyloid antibodies (e.g., anti-human amyloid antibodies and/or anti-heterologous amyloid antibodies). For example, this document provides anti-amyloid antibodies, methods for making anti-amyloid antibodies, and methods for using an anti-amyloid antibody to inhibit amyloid plaques.

In general, one aspect of this document features a substantially pure antibody having binding affinity for human amyloid and a heterologous amyloid. The antibody can be a single chain variable fragment. The antibody can have less than 10⁴ mol⁻¹ binding affinity for monomeric Aβ42. The antibody can have less than two percent cross reactivity with monomeric Aβ42. The antibody can be monoclonal. The antibody can comprise, or consist essentially of, the amino acid sequence set forth in FIG. 4, 5, 6, 7, 8, 9, 10, 11, 12, 13, 14, 15, 16, 17, 18, 19, 25, 26, 27, or 28. The human amyloid can comprise human fibrillar Aβ42. The heterologous amyloid can comprise Sup35-6, AVS12, CS25, CS35, AVS41, AVS6, AVS8, AVS25, Ca Silk, Cb Silk, ccβ, E7A, Sup35-7, or BOC.

In another aspect, this document features a method for inhibiting Aβ plaque formation in a mammal. The method comprises administering an antibody to the mammal, wherein the antibody has binding affinity for human amyloid and a heterologous amyloid. The antibody can be a single chain variable fragment. The antibody can have less than 10⁴ mol⁻¹ binding affinity for monomeric Aβ42. The antibody can have less than two percent cross reactivity with monomeric Aβ42. The antibody can be monoclonal. The antibody can comprise, or consist essentially of, the amino acid sequence set forth in FIG. 4, 5, 6, 7, 8, 9, 10, 11, 12, 13, 14, 15, 16, 17, 18, 19, 25, 26, 27, or 28. The human amyloid can comprise human fibrillar Aβ42. The heterologous amyloid can comprise Sup35-6, AVS12, CS25, CS35, AVS41, AVS6, AVS8, AVS25, Ca Silk, Cb Silk, ccβ, E7A, Sup35-7, or BOC.

In another aspect, this document features a nucleic acid construct comprising, or consisting essentially of, a nucleic acid sequence encoding the amino acid sequence set forth in FIG. 4, 5, 6, 7, 8, 9, 10, 11, 12, 13, 14, 15, 16, 17, 18, 19, 25, 26, 27, or 28. The construct can be an AAV vector.

In another aspect, this document features a substantially pure antibody having binding affinity for an Aβ epitope, wherein the Aβ epitope is the epitope of scFv Pan 89, scFv Pan 34, scFv Pan SUP73, scFv Pan SUP 40, scFv Pan BOC8, scFv Pan SUP 29, scFv Pan 21, scFv Pan 65, scFv Pan 82, scFv Pan 21′, scFv Pan 34′, scFv Pan 65′, scFv Pan 82′, scFv Pan 89′, scFv Pan B8, scFv Pan 29, scFv 4281, scFv 4281-6, scFv 55-1, or scFv 88-1. The antibody can be a single chain variable fragment. The antibody can have less than 10⁴ mol⁻¹ binding affinity for monomeric Aβ42. The antibody can have less than two percent cross reactivity with monomeric Aβ42. The antibody can be monoclonal. The antibody can comprise, or consist essentially of, the amino acid sequence set forth in FIG. 4, 5, 6, 7, 8, 9, 10, 11, 12, 13, 14, 15, 16, 17, 18, 19, 25, 26, 27, or 28.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1. Anti-amyloid Abs were produced by fAβ42 and hAs. (A,B) At day 7, an anti-fAβ42 and anti-amyloid IgM titer (1:500 dilution, measured using a ccβ amyloid plate) was observed following immunization with fAβ42 preparations (fAβ42) and a mixture of hAs (hA mix; 1:1; CSP1-25:Sup35-7). Black bars show reactivity against fAβ42, and gray bars show reactivity against hA ccβ. (B,C) Several hAs induced an anti-Aβ42 amyloid IgM titer. Response was influenced by background of mice. (B) B6/SJL. (D) BALBc. Highest titers were observed with the cold shock polypeptide 1-25+1-35 hA (CS25+35) and hA BOC, a two amino acid amyloid forming dipeptide (Boc-γAbu-mABA-Ome). Seven day sera from a hA BOC vaccinated mouse reacts to multiple amyloids. Reactivity against fAβ42 and hAs AVS41, CS35, Sup35-7, ccβ, and E7 are shown. Only an IgM titer was detected. No IgG titer was present. Similar data were observed with multiple hA immune sera. (E) Sera from HA vaccinated mice recognized plaques. Data are shown for sera from hA AVS immunized mice diluted at 1:100 (middle panel) and 1:200 (bottom panel). Reproducible staining was only observed with an anti-IgM secondary. No staining was seen with a pre-immune sera or secondary alone. Top panel involved using an anti-mAb9 positive control (anti-IgG secondary).

FIG. 2. Anti-amyloid scFVs. (A) Schematic of “panning” for anti-amyloid antibodies. Two rounds of panning are shown. (B) ELISA of putative anti-amyloid scFv phagemids against three amyloids (fAβ42, hA AVS41, and hA AVS 6). An anti-ubiquitin scFv phagemid was used as a control. Clone Pan 49 exhibited strong reactivity with all three amyloids. Other clones exhibited preferential binding of fAβ and hA AVS41. (C) 293T cells were transiently transfected with vectors encoding anti-amyloid scFvs. Secretion of amyloid binding scFvs was assessed by amyloid pulldown and western blotting of the material bound to the amyloid. Two anti-amyloid scFvs ˜30 kDa in MW (Pan34 and Pan82) bind fAβ42 and fAVS25. In this study, several other putative anti-amyloid scFvs isolated from panning a phage library did not appear to bind amyloid in this paradigm. However, at least five additional scFvs do.

FIG. 3 is a graph plotting results showing that anti-amyloid scFvs attenuate Aβ deposition in 3 month old CRND8 mice. Newborn CRND8 mice were injected ICV with AAV1 expressing scFv 21, 34, 82, or 89. Control mice received AAV1-ns scFv ns. Three months later mice were sacrificed following treatment. One hemibrain was used for immunohistochemistry, and the other for biochemical analysis. Total extractable Aβ40 and Aβ42 levels are shown. All anti-amyloid scFvs produced a significant decrease in Aβ40 deposition. There was a trend towards decreased Aβ42 deposition with scFv21. * p<0.05, ** p<0.01 (ANOVA Dunnet's post test).

FIG. 4 (top) is a schematic of the general structure of anti-amyloid scFvs. FIG. 4 (bottom) provides the nucleic acid (SEQ ID NO:1) and amino acid (SEQ ID NO:2) sequences of an anti-amyloid scFv designated Pan 89. The first underlined sequence is a kappa leader sequence, the second underlined sequence is a heavy chain variable region sequence, the third underlined sequence is a heavy chain small variable region, the fourth underlined sequence is a kappa chain small variable region sequence, the fifth underlined sequence is a kappa chain variable region sequence, and the sixth underlined sequence is a his-Myc tag sequence.

FIG. 5 provides the nucleic acid (SEQ ID NO:3) and amino acid (SEQ ID NO:4) sequences of an anti-amyloid scFv designated Pan 34.

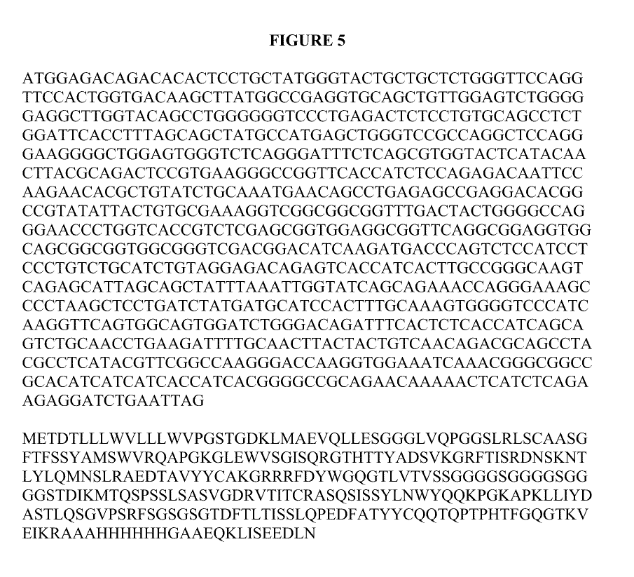

FIG. 6 provides the nucleic acid (SEQ ID NO:5) and amino acid (SEQ ID NO:6) sequences of an anti-amyloid scFv designated Pan SUP73.

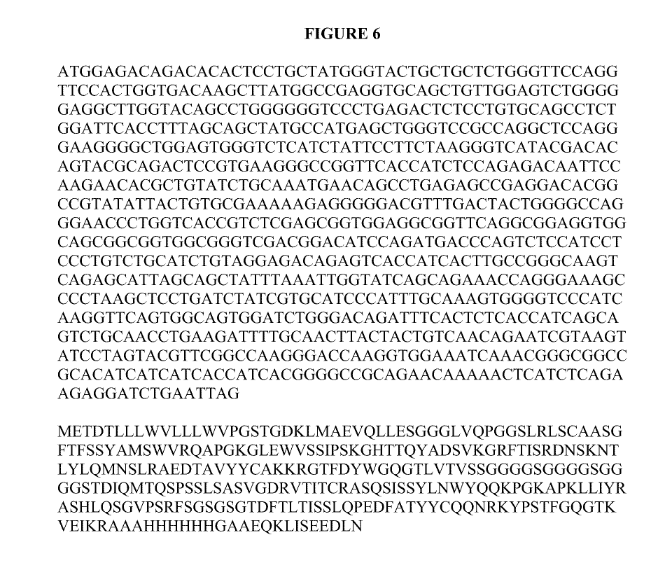

FIG. 7 provides the nucleic acid (SEQ ID NO:7) and amino acid (SEQ ID NO:8) sequences of an anti-amyloid scFv designated Pan SUP 40.

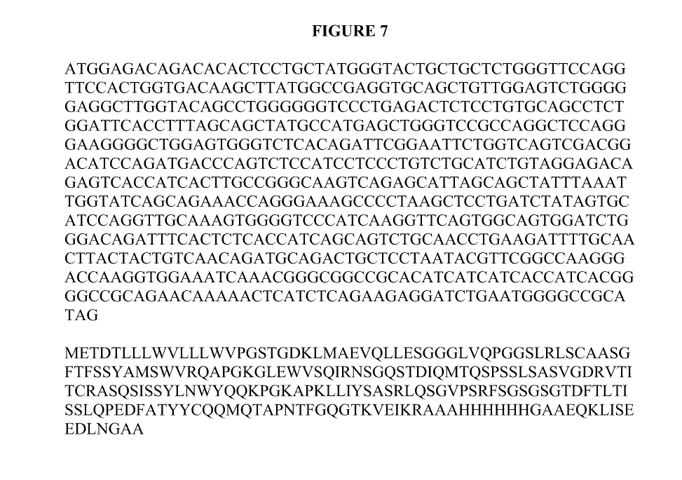

FIG. 8 provides the nucleic acid (SEQ ID NO:9) and amino acid (SEQ ID NO:10) sequences of an anti-amyloid scFv designated Pan BOC8.

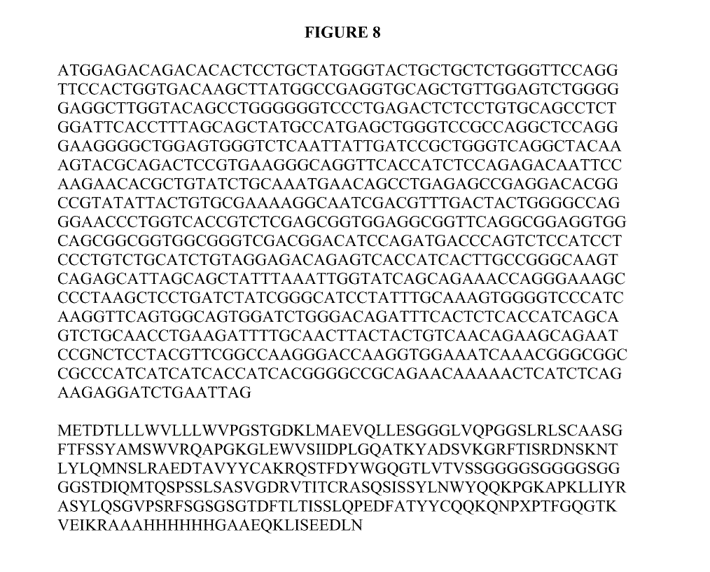

FIG. 9 provides the nucleic acid (SEQ ID NO:11) and amino acid (SEQ ID NO:12) sequences of an anti-amyloid scFv designated Pan SUP 29.

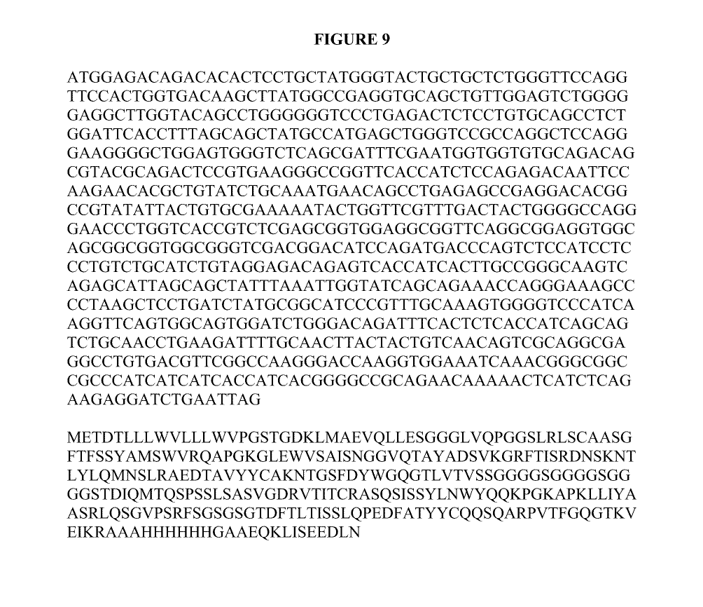

FIG. 10 provides the nucleic acid (SEQ ID NO:13) and amino acid (SEQ ID NO:14) sequences of an anti-amyloid scFv designated Pan 21.

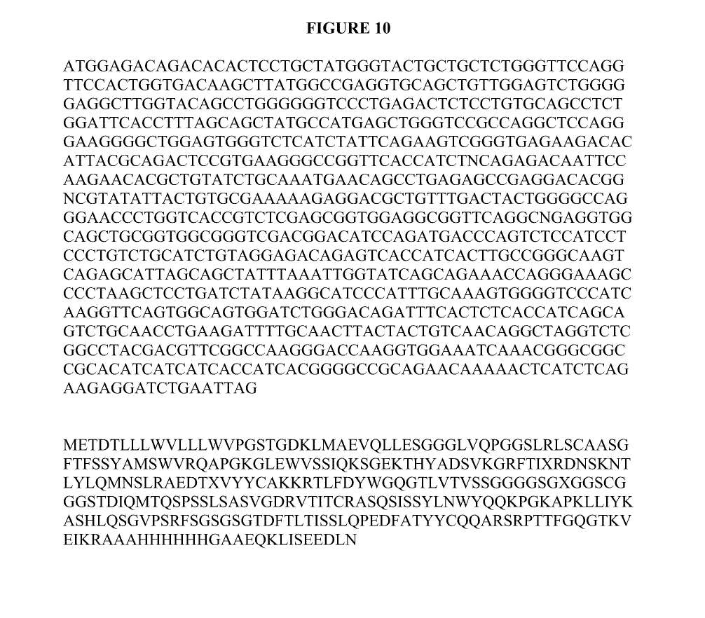

FIG. 11 provides the nucleic acid (SEQ ID NO:15) and amino acid (SEQ ID NO:16) sequences of an anti-amyloid scFv designated Pan 65.

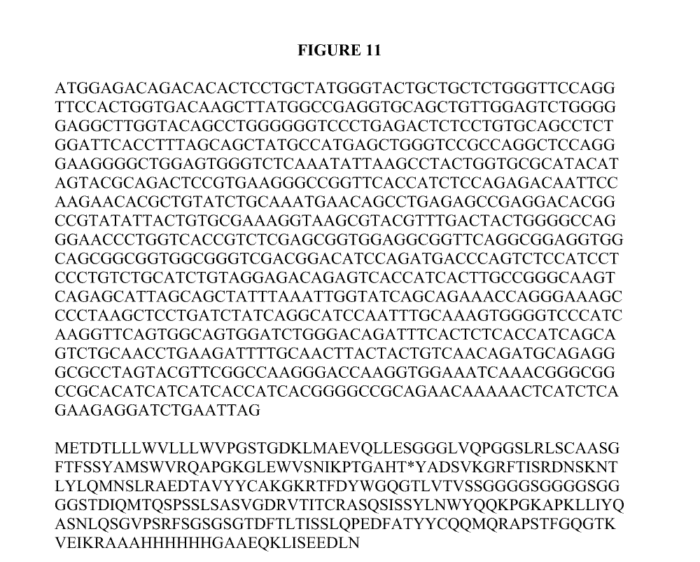

FIG. 12 provides the nucleic acid (SEQ ID NO:17) and amino acid (SEQ ID NO:18) sequences of an anti-amyloid scFv designated Pan 82.

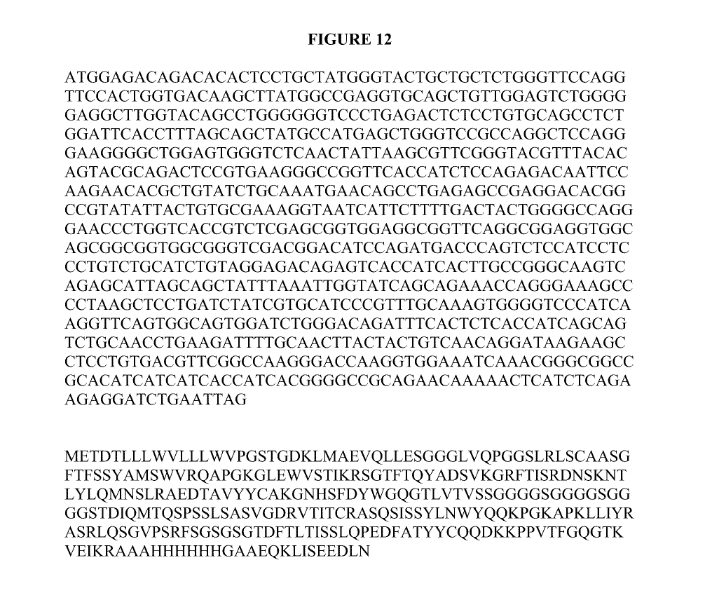

FIG. 13 provides the nucleic acid (SEQ ID NO:81) and amino acid (SEQ ID NO:82) sequences of an anti-amyloid scFv designated Pan 21′.

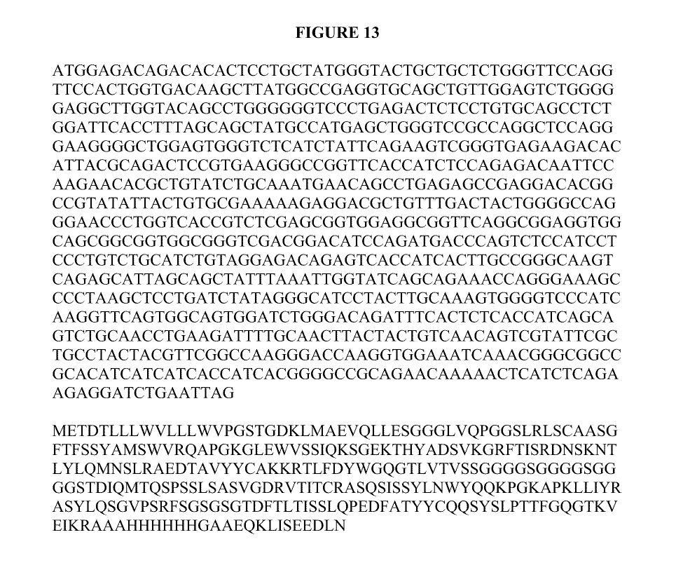

FIG. 14 provides the nucleic acid (SEQ ID NO:48) and amino acid (SEQ ID NO:49) sequences of an anti-amyloid scFv designated Pan 34′.

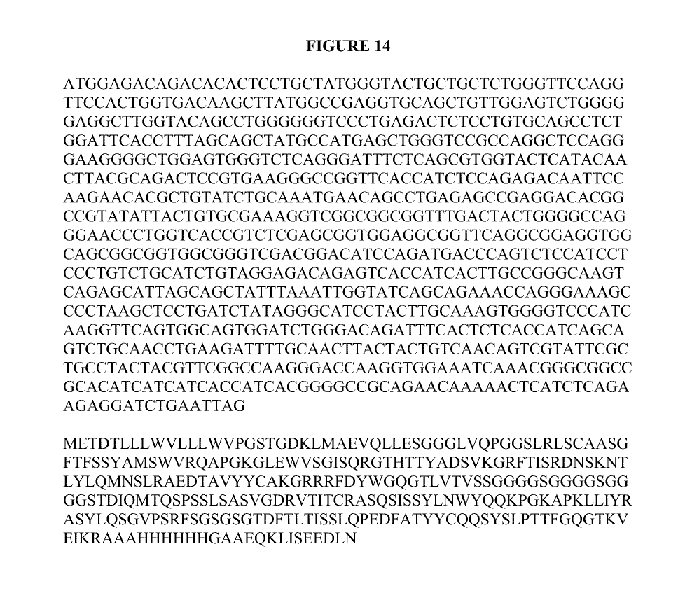

FIG. 15 provides the nucleic acid (SEQ ID NO:50) and amino acid (SEQ ID NO:51) sequences of an anti-amyloid scFv designated Pan 65′.

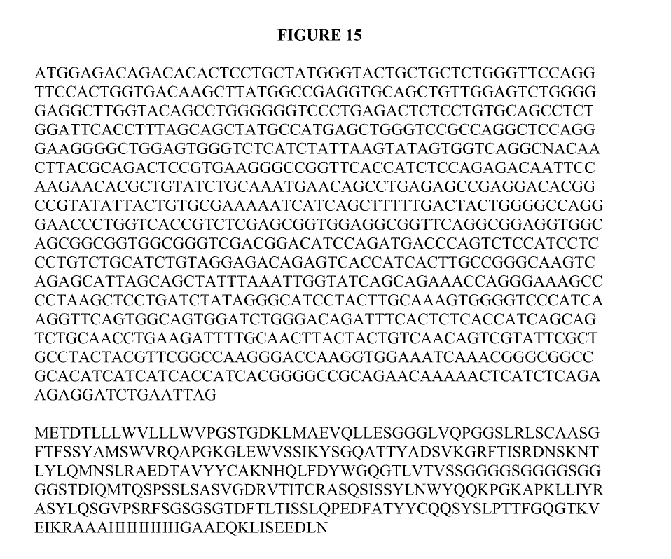

FIG. 16 provides the nucleic acid (SEQ ID NO:52) and amino acid (SEQ ID NO:53) sequences of an anti-amyloid scFv designated Pan 82′.

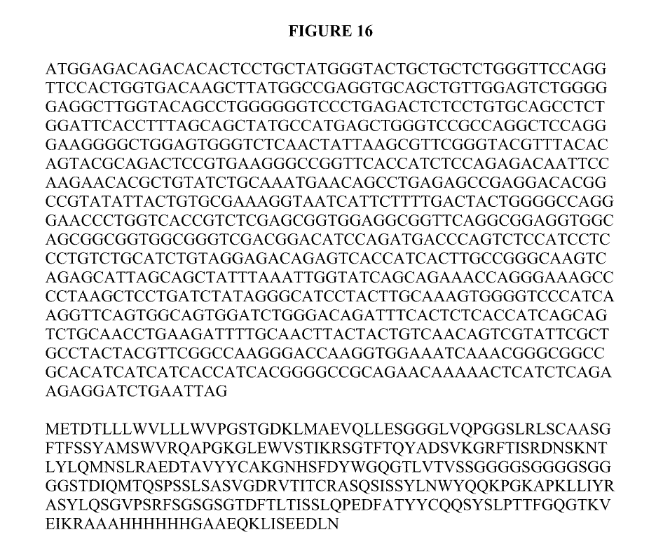

FIG. 17 provides the nucleic acid (SEQ ID NO:54) and amino acid (SEQ ID NO:55) sequences of an anti-amyloid scFv designated Pan 89′.

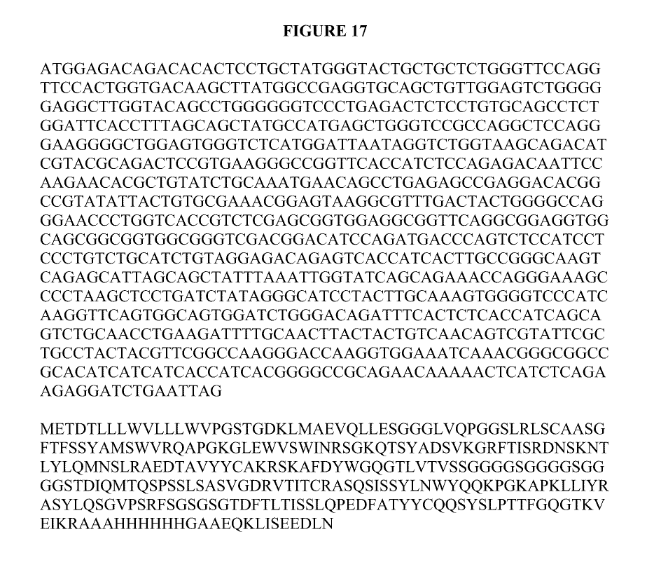

FIG. 18 provides the nucleic acid (SEQ ID NO:56) and amino acid (SEQ ID NO:57) sequences of an anti-amyloid scFv designated Pan B8.

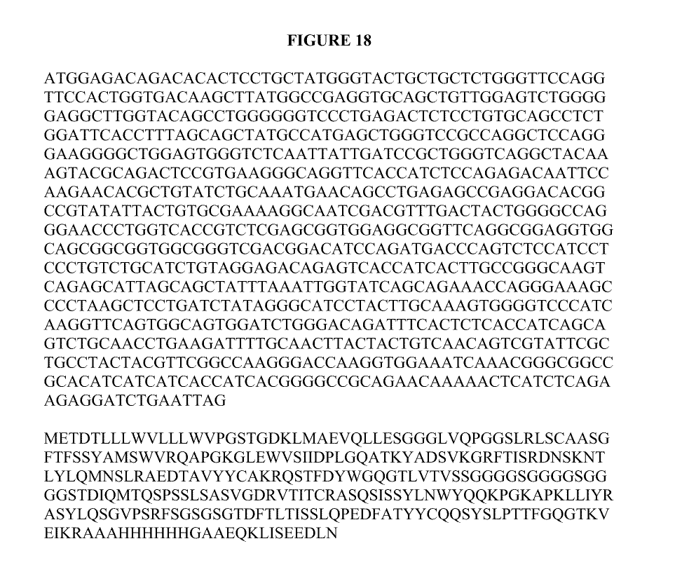

FIG. 19 provides the nucleic acid (SEQ ID NO:58) and amino acid (SEQ ID NO:59) sequences of an anti-amyloid scFv designated Pan 29.

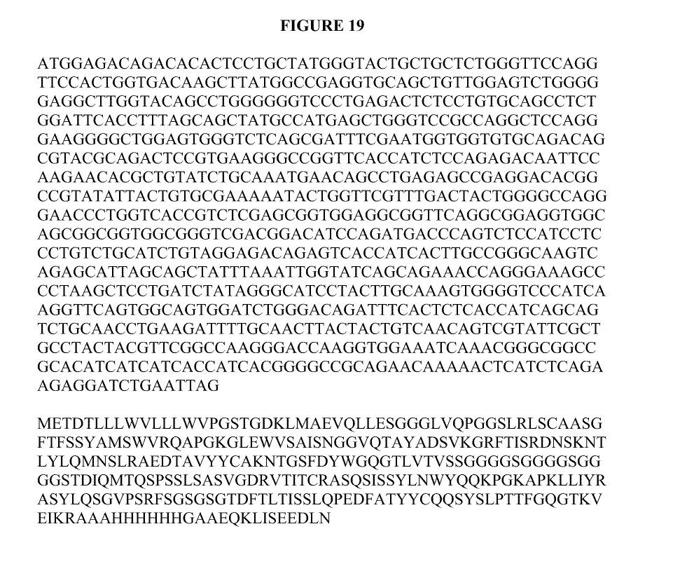

FIG. 20 is a graph of representative ELISA reactivity of putative anti-amyloid scFv phagemids against three amyloids (fAb42, hA AVS41, and hA CS35). Anti-ubiquitin scFv phagemid was used as a control. In this figure, scFv82 refers to an anti-amyloid scFv designated Pan 82′ having the sequence set forth in FIG. 16, scFv89 refers to an anti-amyloid scFv designated Pan 89′ having the sequence set forth in FIG. 17, scFv65 refers to an anti-amyloid scFv designated Pan 65′ having the sequence set forth in FIG. 15, scFv34 refers to an anti-amyloid scFv designated Pan 34′ having the sequence set forth in FIG. 14, and scFv21 refers to an anti-amyloid scFv designated Pan 21′ having the sequence set forth in FIG. 13.

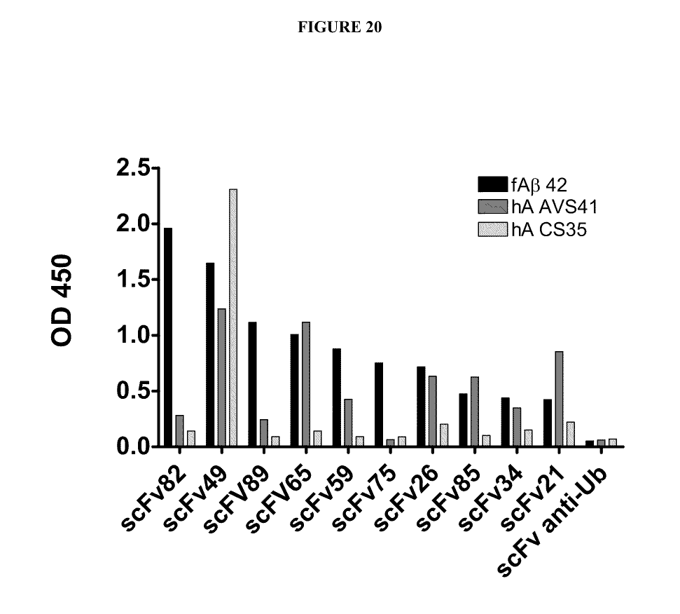

FIG. 21 is a table of scFvs expressed in 293 cells. The sequence of pulldowns used to pan for these scFvs and the “randomized” sequences of the V_{H} and V_{L} regions are shown.

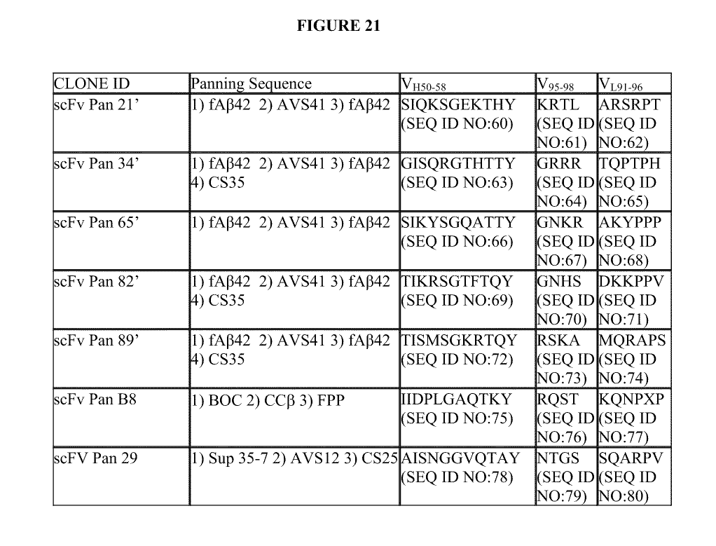

FIG. 22 contains results from a representative amyloid pulldown experiment using conditioned media from stable 293 cells expressing anti-Aβ (scFv9, scFv42.2) and anti-amyloid scFvs (scFv21, scFv82). Aβ amyloid or hA from AVS41, CS35, or BOC polypeptides were used to assess reactivity to amyloid. Ni refers to nickel affinity agarose bead pulldown as a positive control for scFv in the conditioned media. Strept refers to streptavidin agarose bead pulldown used as a control for non-specific binding. In this figure, scFv21 refers to an anti-amyloid scFv designated Pan 21′ having the sequence set forth in FIG. 13, and scFv82 refers to an anti-amyloid scFv designated Pan 82′ having the sequence set forth in FIG. 16.

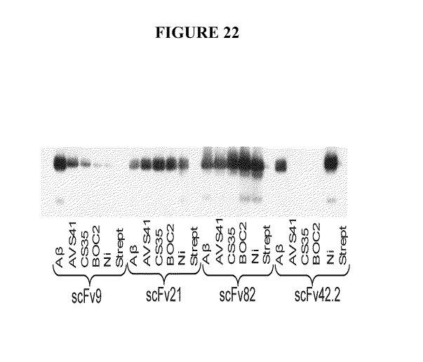

FIG. 23 is a graph of representative ELISA reactivity of anti-Aβ, anti-BSA, and anti-amyloid scFvs (scFv21, scFv82, scFvB8) against plates coated 1 μg/mL monomeric Aβ, SDS oligomer, and Aβ amyloid fibrils. In this figure, scFv21 refers to an anti-amyloid scFv designated Pan 21′ having the sequence set forth in FIG. 13, and scFv82 refers to an anti-amyloid scFv designated Pan 82′ having the sequence set forth in FIG. 16.

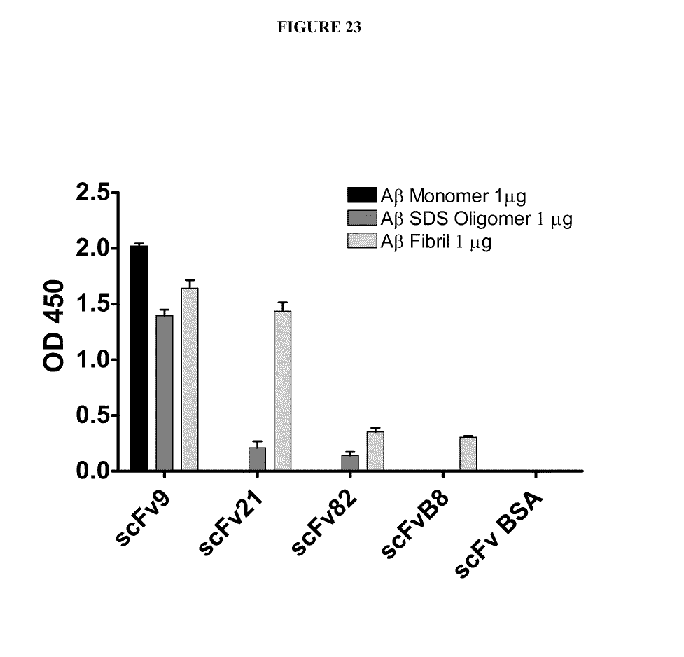

FIG. 24 is a graph plotting Aβ levels in CRND8 mice treated with the indicated scFv. rAAV1 delivery of anti-amyloid scFvs reduced biochemical Aβ loads. In this figure, scFv21 refers to an anti-amyloid scFv designated Pan 21′ having the sequence set forth in FIG. 13, scFv34 refers to an anti-amyloid scFv designated Pan 34′ having the sequence set forth in FIG. 14, scFv82 refers to an anti-amyloid scFv designated Pan 82′ having the sequence set forth in FIG. 16, and scFv89 refers to an anti-amyloid scFv designated Pan 89′ having the sequence set forth in FIG. 17.

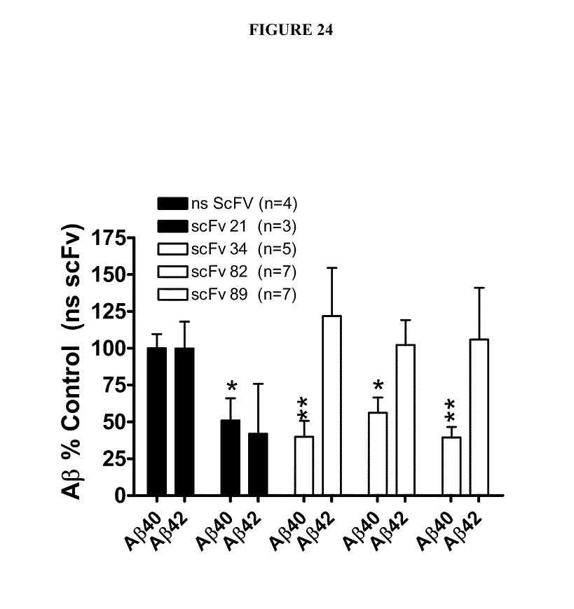

FIG. 25 provides the nucleic acid (SEQ ID NO:85) and amino acid (SEQ ID NO:86) sequences of an anti-amyloid scFv designated scFv 4281.

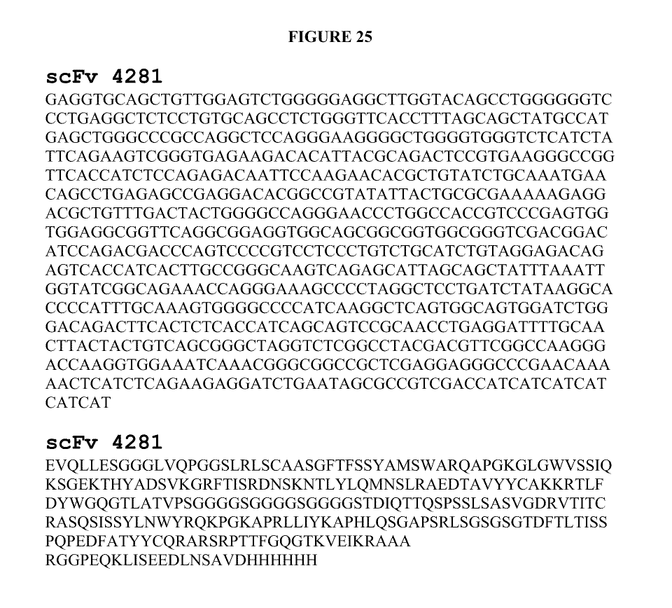

FIG. 26 provides the nucleic acid (SEQ ID NO:87) and amino acid (SEQ ID NO:88) sequences of an anti-amyloid scFv designated scFv 4281-6.

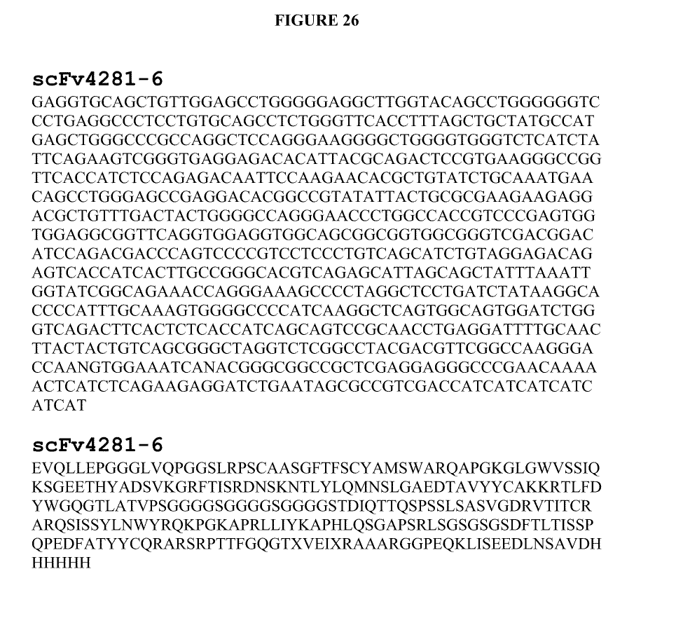

FIG. 27 provides the nucleic acid (SEQ ID NO:89) and amino acid (SEQ ID NO:90) sequences of an anti-amyloid scFv designated scFv 55-1.

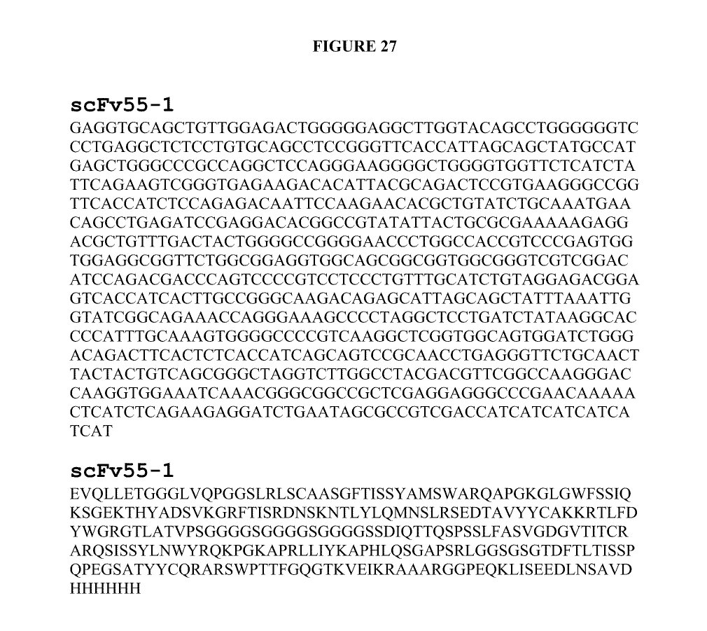

FIG. 28 provides the nucleic acid (SEQ ID NO:91) and amino acid (SEQ ID NO:92) sequences of an anti-amyloid scFv designated scFv 88-1.

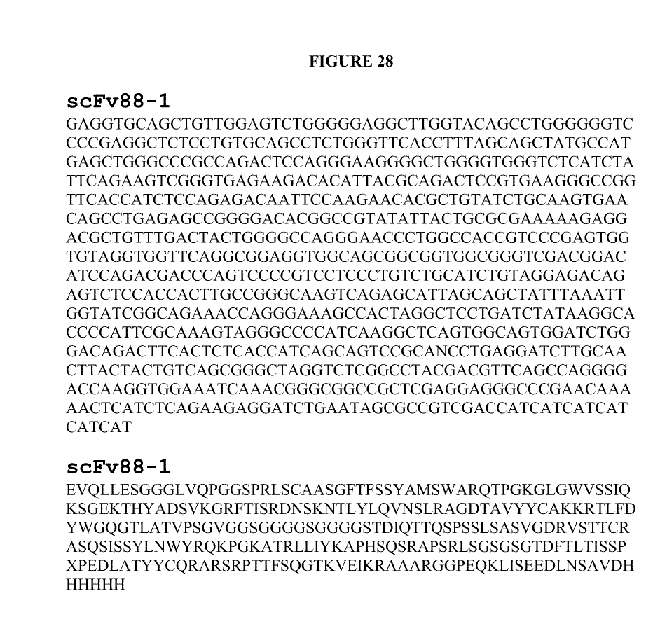

### DETAILED DESCRIPTION

This document provides methods and materials related to anti-amyloid antibodies. For example, this document provides anti-amyloid antibodies, methods for making anti-amyloid antibodies, and methods for using an anti-amyloid antibody to treat or prevent an amyloid condition (e.g., AD). An anti-amyloid antibody is an antibody that recognizes multiple amyloids (e.g., two or more, three or more, four or more, or five or more amyloids) formed from non-homologous polypeptides. Such anti-amyloid antibodies can recognize the conformation of amyloid and not the primary sequence of the polypeptide subunit. In such cases, an anti-amyloid antibody can have a higher avidity for amyloid formed from a polypeptide then the antibody does for the monomeric soluble polypeptide that forms the amyloid aggregate.

As used herein, “amyloidogenic polypeptides” are polypeptides that can form amyloids or pre-amyloid aggregates. Amyloid is an insoluble, ordered aggregate of polypeptides that are fibrillar in structure, and that can be detected by binding to Congo Red or a Thioflavin (e.g., Thioflavin T). Staining conditions for Congo Red and Thioflavins are provided elsewhere (Merlini and Bellotti, 2003, N. Engl. J. Med., 349:583-596; and Glenner, 1980, N. Engl. J. Med., 302:1283-1292). Typically, an amyloid has a diameter of approximately 10 nm with lengths up to several micrometers. Pre-amyloid aggregates are smaller than amyloids (typically less than 200 nm in length), soluble, and structurally resemble a spherical particle, a curvilinear protofibril, or an annular pore. Atomic force microscopy can be used to determine the structure of pre-amyloid aggregates. Amyloidogenic polypeptides can be eight amino acids in length or longer and can have less than 40 percent (e.g., less than 35 percent) identity to any polypeptide from the mammal to receive an antibody provided herein. In some cases, an amyloidogenic polypeptide can contain no more than seven contiguous amino acids (e.g., 6 amino acids or less) of any polypeptide encoded by the genome of the mammal (e.g., a human) to receive an antibody provided herein.

Non-limiting examples of amyloidogenic polypeptides include polypeptides from the amino terminus (residues 1-37) of bacterial cold shock proteins such as a Bacillus subtilis or Bacillus lichenformis major cold shock protein. For example, a suitable polypeptide can contain residues 1-25 of the B. subtilis and B. licheniformis major cold shock protein (MLEGKVKWFNSEKGFGFIEVEG, SEQ ID NO:19) or can contain residues 1-35 of the B. subtilis and B. licheniformis major cold shock protein (MLEGKVKWFNSEKGFGFIEVEGQDDVFVHFSAIQG, SEQ ID NO:20).

Polypeptides from the shaft sequence of human adenovirus fiber proteins also can be used. For example, a suitable polypeptide can contain 6 (GAITIG, SEQ ID NO:21), 8 (NSGAITIG, SEQ ID NO:22), 12 (LSFDNSGAITIG, SEQ ID NO:23), 25 (AMITKLGSGLSFDNSGAITIGNKND, SEQ ID NO:24), or 41 (PIKTKIGSGIDYNENGAMITKLGSGLSFDNSGAITIGNKND, SEQ ID NO:25) amino acids from the shaft region (amino acids 356-396) of the adenovirus type 2 fiber protein.

Other suitable polypeptides can be derived from the chorion class A protein pc292 precursor from Antheraea polyphemus (e.g., a polypeptide having the sequence: SYGGEGIGNVAVAGELPVAGKTAVAGRVPIIGAVGFGGPAGAAGAVSIAGR, SEQ ID NO:26) or chorion protein from Bombyx mori (e.g., a polypeptide having the sequence: GNLPFLGTAXVAGEFPTA, SEQ ID NO:27, where X is G or D). The monellin chain A (FREIKGYEYQLYVYASDKLFRADISEDYKTRGRKLLRFNGPVPPP, SEQ ID NO:28) and the monellin chain B (GEWEIIDIGPFTQNLGKFAVDEENKIGQYGRLTFNKVIRPCMKKTIYEEN, SEQ ID NO:29) proteins from Dioscoreophyllum cumminsii and fragments of the monellin chain A and B proteins also are suitable. Bacterial curlin/CSGA and related proteins, and fragments of such proteins also are useful. Non-limiting examples of such proteins include the curlin/CSGA protein from Escherichia coli (GenBank Accession No. CAA62282.1, GI:1147564, MKLLKVAAIAAIVFSGSALAGVVPQYGGGGNHGGGGNNSGPNSELNIYQYGGGNSALALQT DARNSDLTITQHGGGNGADVGQGSDDSSIDLTQRGFGNSATLDQWNGKNSEMT VKQFGGGNGAADQTASNSSVNVTQVGFGNNATAHQY, SEQ ID NO:30), a curlin subunit from E. coli (GenBank Accession No. AAA23616.1, GI:290425); CsgA protein from E. coli (GenBank Accession No. AAK53212.1, GI:14039401); curlin-csgA protein from Enterobacter sakazakii (GenBank Accession No. CAD56678.1, GI:31790502), Citrobacter freundii (GenBank Accession No. CAD56675.1, GI:31790498), or Citrobacter sp. Fec2 (GenBank Accession No. CAD56672.1, GI:31790494); major curlin subunit precursor from E. coli, e.g., E. coli CFT073 (GenBank Accession No. NP_{—}753219.1, GI:26247179) or E. coli K12 (GenBank Accession No. BAA35840.1, GI:1651514); major curlin subunit precursor from Salmonella enterica (GenBank Accession No. YP_{—}150943.1, GI:56413868), AgfA protein from Salmonella typhimurium (GenBank Accession No. CAA04151.1, GI:2275121, MKLLKVAAFAAIVVSGSAVAGVVPQWGGGGNHNGGGNSSGPDSTLSIYQYGSANAALALQSDARKSET TITQSGYGNGADVGQGADNSTIELTQNGFRNNATIDQWNAKNSDITVGQYGGN NAALVNQTASDSSVMVRQVGFGNNAPANQYN, SEQ ID NO:31); or SEF17 fimbrin protein from Salmonella enteritidis (GenBank Accession No. AAA98671.1, GI:1293678).

The Sup35 protein from Saccharomyces cerevisiae (GenBank Accession No. NP_{—}010457.1, GI:6320377, MSDSNQGNNQQNYQQYSQNGNQQQGNNRYQGYQAYNAQAQPAGGYYQNYQGYSGYQQGGYQQYNPDAGYQQQYNPQGGYQQYN PQGGYQQQFNPQGGRGNYKNFNYNNNLQGYQAGFQPQSQGMSLNDFQKQQKQ AAPKPKKTLKLVSSSGIKLANATKKVGTKPAESDKKEEEKSAETKEPTKEPTKVE EPVKKEEKPVQTEEKTEEKSELPKVEDLKISESTHNTNNANVTSADALIKEQEEE VDDEVVNDMFGGKDHVSLIFMGHVDAGKSTMGGNLLYLTGSVDKRTIEKYERE AKDAGRQGWYLSWVMDTNKEERNDGKTIEVGKAYFETEKRRYTILDAPGHKM YVSEMIGGASQADVGVLVISARKGEYETGFERGGQTREHALLAKTQGVNKMVV VVNKMDDPTVNWSKERYDQCVSNVSNFLRAIGYNIKTDVVFMPVSGYSGANLK DHVDPKECPWYTGPTLLEYLDTMNHVDRHINAPFMLPIAAKMKDLGTIVEGKIE SGHIKKGQSTLLMPNKTAVEIQNIYNETENEVDMAMCGEQVKLRIKGVEEEDISP GFVLTSPKNPIKSVTKFVAQIAIVELKSIIAAGFSCVMHVHTAIEEVHIVKLLHKLE KGTNRKSKKPPAFAKKGMKVIAVLETEAPVCVETYQDYPQLGRFTLRDQGTTIAI GKIVKIAE, SEQ ID NO:32) or the Ure2p protein from Saccharomyces cerevisiae (GenBank Accession No. AAM93191) can be used as amyloidogenic polypeptides as well as fragments of the Sup35 and Ure2p proteins. In addition, Sup35 and Ure2p related proteins and fragments of such proteins can be used. Suitable Sup35 related proteins include, for example, translation release factor 3 from Candida albicans (GenBank Accession No. AAB82541.1, GI:2582369); polypeptide release factor 3 from Zygosaccharomyces rouxii (GenBank Accession No. BAB12684.2, GI:13676384), Candida maltosa (GenBank Accession No. BAB12681.2, GI:13676380), or Debaryomyces hansenii (GenBank Accession No. BAB12682.3, GI:15080702); a protein product from Candida glabrata CBS138 (GenBank Accession No. CAG58641.1, GI:49525028), Kluyveromyces lactis NRRL Y-1140 (GenBank Accession No. CAH00927.1, GI:49642965), or Debaryomyces hansenii CBS767 (GenBank Accession No. CAG85369.1, GI:49653030); SUP35 homolog from Zygosaccharomyces rouxii (GenBank Accession No. AAF14007.1, GI:6478796), Kluyveromyces lactis (GenBank Accession No. AAF14003.1, GI:6478792), Kluyveromyces marxianus (GenBank Accession No. AAF14004.1 GI:6478793), Saccharomycodes ludwigii (GenBank Accession No. AAF14006.1, GI:6478795), or Pichia pastoris (GenBank Accession No. AAF14005.1, GI:6478794); AGL145W protein from Ashbya gossypii (GenBank Accession No. AAS54346.1, GI:44985722); and EF-1alpha-like protein factor from Pichia pinus (GenBank Accession No. CAA40231.1, GI:3236).

Alanine rich antifreeze polypeptides also can be used as amyloidogenic polypeptides. For example, antifreeze polypeptide SS-3 (GenBank Accession No. P04367, GI:113894, MNAPARAAAKTAADALAAAKKTAADAAAAAAAA, SEQ ID NO:33) and related polypeptides can be used. Non-limiting examples of SS-3 related polypeptides include antifreeze sculpin polypeptide (GenBank Accession No. 1Y04_A, GI:62738562); antifreeze polypeptide GS-5 (GenBank Accession No. P20421, GI:113904); longhorn sculpin skin-type antifreeze protein from Myoxocephalus octodecemspinosus (GenBank Accession No. AAG22048.1, GI:10717168); antifreeze protein 3—winter flounder (Pseudopleuronectes americanus) (GenBank Accession No. FDFL3W, GI:72032); Afa5 antifreeze protein from tobacco (GenBank Accession No. AAB20142.1, GI:237857); Type I antifreeze protein from Prochlorococcus marinus (GenBank Accession No. CAE21324.1, GI:33640869); antifreeze protein—winter flounder (GenBank Accession No. I51125, GI:2134023, GenBank Accession No. JS0705, GI:85670, GenBank Accession No. CAA30389.1, GI:64212, GenBank Accession No. AAA49472.1, GI:213595 and GenBank Accession No. 1212275A, GI:225327); Afa3antifreeze protein from tobacco (GenBank Accession No. AAB20141.1, GI:237856); antifreeze protein SS-8—shorthorn sculpin (GenBank Accession No. A05163, GI:85611); Antifreeze protein A/B precursor (P04002, GI:113914); antifreeze polypeptide GS-8 from Myoxocephalus aenaeus (GenBank Accession No. P20617, GI:113909); synthetic flounder antifreeze protein (GenBank Accession No. AAA72967.1, GI:554531); chain B antifreeze protein from winter flounder (GenBank Accession No. 1WFB_B GI:1065084); skin-type antifreeze polypeptide AFP-2 from Myoxocephalus scorpius (GenBank Accession No. AAG25982.1, GI:10998655); membrane spanning protein from Shigella flexneri (GenBank Accession No. NP_{—}706495.1, GI:24111985), E. coli O157:H7 EDL933 (GenBank Accession No. AAG55075.1, GI:12513672), or E. coli K12 (GenBank Accession No. NP_{—}415267.1, GI:16128714); antifreeze prepropeptide from winter flounder (GenBank Accession No. AAB59964.1, GI:457351); putative secreted protein from Streptomyces coelicolor (GenBank Accession No. CAB36606.1, GI:4455743 or GenBank Accession No. CAB62715.1, GI:6562784); COG3144, flagellar hook-length control protein from Burkholderia fungorum (GenBank Accession No. ZP_{—}00278986.1, GI:48782457); CG16779-PA (GenBank Accession No. AAF54383.1, GI:7299186) or CG7434-PA (GenBank Accession No. NP_{—}477134.1, GI:17137152) from Drosophila melanogaster; ribosomal protein L22 from Drosophila melanogaster (GenBank Accession No. AAD19341.1, GI:4378008); Flag-tag_beta-lactamase_tolA fusion protein (GenBank Accession No. AAQ93652.1, GI:37575400); antifreeze protein AFP homolog (GenBank Accession No. AAC60714.1, GI:560670); transcriptional activator from Cryptococcus neoformans (GenBank Accession No. AAW40728.1, GI:57222684); TolA protein from E. coli CFT073 (GenBank Accession No. NP_{—}752748.1, GI:26246708); tol protein from Salmonella typhimurium LT2 (GenBank Accession No. AAL19691.1, GI:16419257), Mapkap1 protein from Mus musculus (GenBank Accession No. AAH48870.1, GI:28981397); protein associated to the polyhydroxyalkanoate inclusion from Pseudomonas sp. 61-3 (GenBank Accession No. BAB91367.1, GI:20502373); CG11203-PA from Drosophila melanogaster (GenBank Accession No. NP_{—}572666.1, GI:24641144); a predicted protein from Magnaporthe grisea 70-15 (GenBank Accession No. EAA50560.1, GI:38103924); ENSANGP00000012554 from Anopheles gambiae str. PEST (GenBank Accession No. EAA11004.2 GI:30175902); Om(1D) from Drosophila ananassae (GenBank Accession No. CAA40011.1, GI:7147); BarH1 from Drosophila ananassae (GenBank Accession No. AAA28381.1, GI:156976); polyhydroxyalkanoate granule-associated protein PhaF from Pseudomonas syringae pv. tomato str. DC3000 (GenBank Accession No. NP_{—}794878.1, GI:28872259); chain B reverse gyrase from Archaeoglobus fulgidus (GenBank Accession No. 1GKU_B, GI:20149845); protein product from Kluyveromyces lactis (GenBank Accession No. CAG99118.1, GI:49643166), Tetraodon nigroviridis (GenBank Accession No. CAF91831.1 GI:47213557), or Limanda ferruginea (GenBank Accession No. CAA29655.1, GI:64042); SD05989p from Drosophila melanogaster (GenBank Accession No. AAM52764.1, GI:21483578); CG7518-PB, isoform A (GenBank Accession No. AAF54888.2, GI:10726500) and isoform B (GenBank Accession No. AAN14338.1, GI:23175967) from Drosophila melanogaster; CG5529-PA from Drosophila melanogaster (GenBank Accession No. NP_{—}523387.1, GI:17737357); radial spoke protein 2 from Chlamydomonas reinhardtii (GenBank Accession No. AAQ92371.1, GI:37528882); OmpA/MotB domain from Rhodopseudomonas palustris (GenBank Accession No. NP_{—}947119.1 GI:39934843), polyhydroxyalkanoate synthesis protein PhaF from Pseudomonas aeruginosa (GenBank Accession No. NP_{—}253747.1, GI:15600253); exodeoxyribonuclease V, predicted protein from Gallus gallus (GenBank Accession No. XP_{—}424728.1, GI:50761474); and COG2913, small protein A (tmRNA-binding) protein from Burkholderia cepacia (GenBank Accession No. ZP_{—}00216624.1, GI:46316044). Other suitable SS-3 related polypeptides include the following hypothetical proteins: BPSS2166 from Burkholderia pseudomallei (GenBank Accession No. YP_{—}112167.1, GI:53723182), Rsph03002275 from Rhodobacter sphaeroides (GenBank Accession No. ZP_{—}00006323.2, GI:46192645), Mdeg02001428 from Microbulbifer degradans (GenBank Accession No. ZP_{—}00317244.1, GI:48863350), VNG0441H from Halobacterium sp. NRC-1 (GenBank Accession No. NP_{—}279507.1, GI:15789683), XP_{—}579923 from Rattus norvegicus (GenBank Accession No. XP_{—}579923.1, GI:62640396), hypothetical protein from Streptomyces coelicolor A3(2) (GenBank Accession No. CAA19786.1, GI:3288614), surface protein from Bacteroides thetaiotaomicron VPI-5482 (GenBank Accession No. AAO76619.1, GI:29338820), RPA4347 from Rhodopseudomonas palustris CGA009 (GenBank Accession No. NP_{—}949683.1 GI:39937407), UM03989.1 from Ustilago maydis (GenBank Accession No. EAK84999.1, GI:46099766), hypothetical protein 4 (phaC2 3′ region) from Pseudomonas aeruginosaor (GenBank Accession No. S29309, GI:485464), CNBH0920 from Cryptococcus neoformans (GenBank Accession No. EAL19399.1, GI:50256676), gp58 from Burkholderia cenocepacia phage BcepB1A (GenBank Accession No. YP_{—}024894.1 GI:48697536), and Oryza sativa (japonica cultivar-group) (GenBank Accession No. BAD61824.1, GI:54291151).

Other suitable polypeptides include fragments of the HET-s protein from Podospora anserine such as GNNQQNY (SEQ ID NO:34) or a fungal hydrophobin polypeptide (e.g., RodA from Aspergillus niger, GenBank Accession No. AAX21520, GI 60476801; Q9UVI4, a trihydrophobin precursor from Claviceps fusiformis, GenBank Accession No. Q9UVI4, GI:25091421; hydrophobin 3 precursor from Agaricus bisporus, GenBank Accession No. O13300, GI 12643535; hydrophobin II precursor from Hypocrea jecorina (GenBank Accession No. P79073, GI 6647555), Pisolithus tinctorius (GenBank Accession No. P52749, GI:1708380), or Agaricus bisporus (GenBank Accession No. P49073, GI 1708379); hydrophobin-like protein ssgA precursor from Metarhizium anisopliae, GenBank Accession No. P52752, GI 1711536; hydrophobin-like protein MPG1 precursor from Magnaporthe grisea, GenBank Accession No. P52751, GI 1709085; hydrophobin I precursor from Hypocrea jecorina (GenBank Accession No. P52754; GI 1708378) or Pisolithus tinctorius (GenBank Accession No. P52748, GI 1708377); spore-wall fungal hydrophobin dewA precursor from Emericella nidulans, GenBank Accession No. P52750, GI 1706367; cryparin precursor from Cryphonectria parasitica, GenBank Accession No. P52753, GI 1706154; hydrophobin 1 from Heterobasidion annosum, GenBank Accession No. ABA46363, GI 76563862; hydrophobin 2 from Heterobasidion annosum, GenBank Accession No. ABA46362, GI 76563860; UM05010.1, a hypothetical protein from Ustilago maydis (GenBank Accession No. XP_{—}761157, GI 71021853) or Caenorhabditis elegans (GenBank Accession No. AAA81483, GI 29570473); rodlet protein precursor from Aspergillus nidulans, GenBank Accession No. XP_{—}682072, GI 67903632; spore-wall hydrophobin precursor from Aspergillus nidulans, GenBank Accession No. XP_{—}681275, GI 67902038; hydrophobin precursor from Neurospora crassa, GenBank Accession No. Q04571, GI 416771; or magnaporin from Magnaporthe grisea, GenBank Accession No. AAD18059, GI 4337063). Other examples of useful polypeptides include a chaplin from Streptomycetes spp. and related polypeptides (e.g., a small membrane protein from Streptomyces coelicolor (GenBank Accession No. NP_{—}625950.1, GI:21220171, or Accession No. NP_{—}626950, GI 21221171) or Thermobifida fusca (GenBank Accession No. YP_{—}290942, GI 72163285); a secreted protein from Streptomyces avermitilis (GenBank Accession No. NP_{—}827811.1, GI:29833177), Streptomyces coelicolor (GenBank Accession No. NP_{—}625949.1, GI:21220170; NP_{—}733581, GI 32141179; AAM78434, GI 21902161; NP_{—}626070, GI 21220291; or NP_{—}631313, GI 21225534); or Streptomyces avermitilis (GenBank Accession No. NP_{—}827812, GI 29833178; NP_{—}822405, GI 29827771; or NP_{—}827654, GI 29833020); a membrane protein from Streptomyces coelicolor, GenBank Accession No. NP_{—}626939, GI 21221160; or a protein from Streptomyces verticillus, GenBank Accession No. AAG43514, GI 12003276. Flagellar basal body protein from Salmonella such as FlgB, FlgC, FlgG, and FliE (GenBank Accession Nos. BAA21014, YP_{—}150913, P16323, and P26462, respectively) or fragments of such flagellar basal body proteins also can be used.

The term “antibody” as used herein refers to intact antibodies as well as antibody fragments that retain some ability to bind an epitope. Such fragments include, without limitation, Fab, F(ab′)2, and Fv antibody fragments. The term “epitope” refers to an antigenic determinant on an antigen to which the paratope of an antibody binds. Epitopic determinants usually consist of chemically active surface groupings of molecules (e.g., amino acid or sugar residues) and usually have specific three dimensional structural characteristics as well as specific charge characteristics.

The antibodies provided herein can be any monoclonal or polyclonal antibody having specific binding affinity for amyloid as opposed to the individual polypeptide subunits of amyloid. Such antibodies can be used in immunoassays in liquid phase or bound to a solid phase. For example, the antibodies provided herein can be used in competitive and non-competitive immunoassays in either a direct or indirect format. Examples of such immunoassays include the radioimmunoassay (RIA) and the sandwich (immunometric) assay. In some cases, the antibodies provided herein can be used to treat or prevent amyloid conditions (e.g., AD). For example, an antibody provided herein can be conjugated to a membrane transport sequence to form a conjugate that can be administered to cells in vitro or in vivo. Examples of membrane transport sequences include, without limitation, AALALPAVLLALLAP (SEQ ID NO:83) (Rojas et al., J Biol Chem, 271(44):27456-61 (1996)) and KGEGAAVLLPVLLAAPG (SEQ ID NO:84) (Zhao et al., Apoptosis, 8(6):631-7 (2003) and Zhao et al., Drug Discov Today, 10(18):1231-6, (2005)). Nucleic acids encoding these membrane transport sequences can be readily designed by those of ordinary skill in the art.

Antibodies provided herein can be prepared using any method. For example, any substantially pure amyloid (e.g., human amyloid or a heterologous amyloid), or fragment thereof, can be used as an immunogen to elicit an immune response in an animal such that specific antibodies are produced. Thus, human fibrillar Aβ42 can be used as an immunizing antigen. In addition, the immunogen used to immunize an animal can be chemically synthesized or derived from translated cDNA. Further, the immunogen can be conjugated to a carrier polypeptide, if desired. Commonly used carriers that are chemically coupled to an immunizing polypeptide include, without limitation, keyhole limpet hemocyanin (KLH), thyroglobulin, bovine serum albumin (BSA), and tetanus toxoid.

In some case, anti-amyloid antibodies can be obtained from a library. For example, a phage display library designed to contain different scFv fragments cloned into phagemid vectors can be screened to obtain anti-amyloid antibodies using panning techniques such as those described herein. In some cases, a panning method can include panning phage display libraries expressing scFv sequentially against multiple distinct amyloids formed from polypeptides that lack primary sequence homology. After the final pan, scFV that bind Aβ amyloid can be identified using a standard ELISA against fibrillar Aβ.

The preparation of polyclonal antibodies is well-known to those skilled in the art. See, e.g., Green et al., Production of Polyclonal Antisera, in IMMUNOCHEMICAL PROTOCOLS (Manson, ed.), pages 1 5 (Humana Press 1992) and Coligan et al., Production of Polyclonal Antisera in Rabbits, Rats, Mice and Hamsters, in CURRENT PROTOCOLS IN IMMUNOLOGY, section 2.4.1 (1992). In addition, those of skill in the art will know of various techniques common in the immunology arts for purification and concentration of polyclonal antibodies, as well as monoclonal antibodies (Coligan, et al., Unit 9, Current Protocols in Immunology, Wiley Interscience, 1994).

The preparation of monoclonal antibodies also is well-known to those skilled in the art. See, e.g., Kohler & Milstein, Nature 256:495 (1975); Coligan et al., sections 2.5.1 2.6.7; and Harlow et al., ANTIBODIES: A LABORATORY MANUAL, page 726 (Cold Spring Harbor Pub. 1988). Briefly, monoclonal antibodies can be obtained by injecting mice with a composition comprising an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well established techniques. Such isolation techniques include affinity chromatography with Protein A Sepharose, size exclusion chromatography, and ion exchange chromatography. See, e.g., Coligan et al. , sections 2.7.1 2.7.12 and sections 2.9.1 2.9.3; Barnes et al., Purification of Immunoglobulin G (IgG), in METHODS IN MOLECULAR BIOLOGY, VOL. 10, pages 79 104 (Humana Press 1992).

In addition, methods of in vitro and in vivo multiplication of monoclonal antibodies is well known to those skilled in the art. Multiplication in vitro can be carried out in suitable culture media such as Dulbecco's Modified Eagle Medium or RPMI 1640 medium, optionally replenished by mammalian serum such as fetal calf serum, or trace elements and growth sustaining supplements such as normal mouse peritoneal exudate cells, spleen cells, and bone marrow macrophages. Production in vitro provides relatively pure antibody preparations and allows scale up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells (e.g., osyngeneic mice) to cause growth of antibody producing tumors. Optionally, the animals are primed with a hydrocarbon, especially oils such as pristane (tetramethylpentadecane) prior to injection. After one to three weeks, the desired monoclonal antibody is recovered from the body fluid of the animal.

In some cases, the antibodies provided herein can be made using non-human primates. General techniques for raising therapeutically useful antibodies in baboons can be found, for example, in Goldenberg et al., International Patent Publication WO 91/11465 (1991) and Losman et al., Int. J. Cancer, 46:310 (1990).

In some cases, the antibodies can be humanized monoclonal antibodies. Humanized monoclonal antibodies can be produced by transferring mouse complementarity determining regions (CDRs) from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions when treating humans. General techniques for cloning murine immunoglobulin variable domains are described, for example, by Orlandi et al., Proc. Nat'l. Acad. Sci. USA, 86:3833 (1989). Techniques for producing humanized monoclonal antibodies are described, for example, by Jones et al., Nature, 321:522 (1986); Riechmann et al., Nature, 332:323 (1988); Verhoeyen et al., Science, 239:1534 (1988); Carter et al., Proc. Nat'l. Acad. Sci. USA, 89:4285 (1992); Sandhu, Crit. Rev. Biotech., 12:437 (1992); and Singer et al., J. Immunol., 150:2844 (1993).

Antibodies provided herein can be derived from human antibody fragments isolated from a combinatorial immunoglobulin library. See, for example, Barbas et al., METHODS: A COMPANION TO METHODS IN ENZYMOLOGY, VOL. 2, page 119 (1991) and Winter et al., Ann. Rev. Immunol., 12: 433 (1994). Cloning and expression vectors that are useful for producing a human immunoglobulin phage library can be obtained, for example, from STRATAGENE Cloning Systems (La Jolla, Calif.). In addition, antibodies provided herein can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens and can be used to produce human antibody secreting hybridomas. Methods for obtaining human antibodies from transgenic mice are described by Green et al., Nature Genet., 7:13 (1994); Lonberg et al., Nature, 368:856 (1994); and Taylor et al., Int. Immunol., 6:579 (1994).

Antibody fragments can be prepared by proteolytic hydrolysis of an intact antibody or by the expression of a nucleic acid encoding the fragment. Antibody fragments can be obtained by pepsin or papain digestion of intact antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. In some cases, an enzymatic cleavage using pepsin can be used to produce two monovalent Fab′ fragments and an Fc fragment directly. These methods are described, for example, by Goldenberg (U.S. Pat. Nos. 4,036,945 and 4,331,647). See, also, Nisonhoff et al., Arch. Biochem. Biophys., 89:230 (1960); Porter, Biochem. J., 73:119 (1959); Edelman et al., METHODS IN ENZYMOLOGY, VOL. 1, page 422 (Academic Press 1967); and Coligan et al. at sections 2.8.1 2.8.10 and 2.10.1 2.10.4.

Other methods of cleaving antibodies, such as separation of heavy chains to form monovalent light heavy chain fragments, further cleavage of fragments, or other enzymatic, chemical, or genetic techniques may also be used provided the fragments retain some ability to bind (e.g., selectively bind) its epitope.

The antibodies provided herein can be substantially pure. The term “substantially pure” as used herein with reference to an antibody means the antibody is substantially free of other polypeptides, lipids, carbohydrates, and nucleic acid with which it is naturally associated in nature. Thus, a substantially pure antibody is any antibody that is removed from its natural environment and is at least 60 percent pure. A substantially pure antibody can be at least about 65, 70, 75, 80, 85, 90, 95, or 99 percent pure.

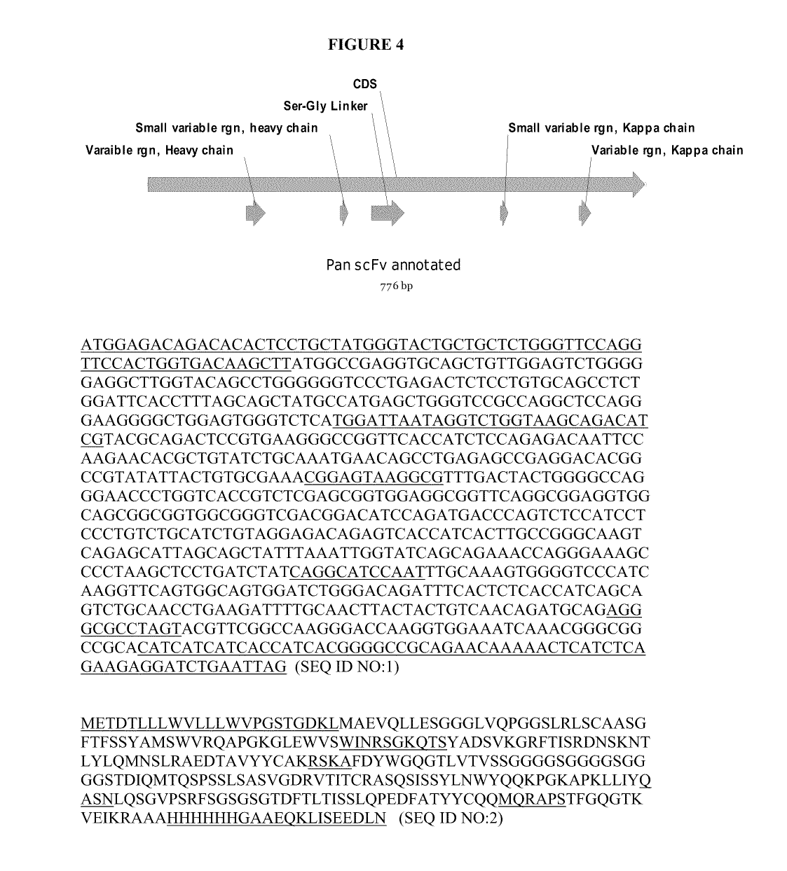

An antibody provided herein can be administered to a mammal under conditions that reduce amyloid aggregates within the mammal or prevent the formation of amyloid aggregates within the mammal. For example, an antibody having the amino acid sequence set forth in FIG. 4, 5, 6, 7, 8, 9, 10, 11, or 12 can be administered to a mammal (e.g., a human). In some cases, nucleic acid encoding an antibody having the amino acid sequence set forth in FIG. 4, 5, 6, 7, 8, 9, 10, 11, or 12 can be administered to a mammal (e.g., a human). Such nucleic acid can be incorporated into a viral vector such as an adenovirus vector.

In some case, the antibodies provided herein can be used to form antibody oligomers. For example, two, three, four, five, or more antibodies (e.g., scFv Pan 21′ antibodies) can be linked to form a single large molecule with multiple paratopes. In some cases, each paratope of an antibody oligomer can be the same. For example, an antibody oligomer can be a molecule having two scFv Pan 21′ antibodies linked together (e.g., covalently linked together). In some cases, an antibody oligomer can contain antibodies with different paratopes. For example, an antibody oligomer can be a molecule having an scFv Pan 21′ antibody linked to an scFv Pan 89′ antibody.

The invention will be further described in the following examples, which do not limit the scope of the invention described in the claims.

### Examples

### Example 1

### Heterologous Amyloids (hAs)

The published literature was searched to identify polypeptides that form amyloid but lack homology to human or mouse polypeptides. The overall homology of published subunit amyloid polypeptides was assessed against all known and predicted mouse and human polypeptides using the BLASTp program algorithm. Polypeptides with <40% overall homology to any human or mouse polypeptides were chosen for further analysis. The BLASTp algorithm that looks for short regions of highly conserved amino acids was used, and choices were limited to polypeptides that contain less then six contiguous amino acids homologous to any human or mouse polypeptide. The rationale for this second screen is to limit potential autoimmune activation. MHC class I can bind polypeptides of eight amino acids in length. Thus, even if overall homology of a polypeptide is not significant, a short region of conserved sequence of a polypeptide can enhance the potential for harmful T-cell activation. Finally, choices were limited to polypeptides of ˜50 amino acids or less in length, as these polypeptides could be easily synthesized in sufficient quantities. Thus, in these initial studies, larger polypeptides capable of forming amyloid were not evaluated. For polypeptides smaller than eight amino acids, the stringency on overall homology was relaxed as these polypeptides are theoretically too small to bind MHC; induction of autoreactive T-cells should not occur. A list of identified polypeptides is provided in Table 1, along with a summary of the characterization.

TABLE 1
hA Polypeptides

| hA | | | | | |
|---|---|---|---|---|---|
| polypeptides | | | | | |
| reference | Sequence (NH2-COOH) | IS | CR | TI | EM |
| AVS6 (1) | GAITIG | X | X | X | IP |
| | (SEQ ID NO: 35) | | | | |
| AVS8 (1) | NSGAITIG | X | X | X | IP |
| | (SEQ ID NO: 36) | | | | |
| AVS12 (1) | LSFDNSGAITIG | X | X | X | IP |
| | (SEQ ID NO: 37) | | | | |
| AVS25 (1) | AMITKLGSGLSFDNSGAITIGNKND | X | X | X | IP |
| | (SEQ ID NO: 38) | | | | |
| AVS41 (1) | PIKTKIGSGIDYNENGAMITKLGSGLSFD | X | X | X | X |
| | NSGAITIGNKND | | | | |
| | (SEQ ID NO: 39) | | | | |
| CS 25 (2, 3) | MLEGKVKWFNSEKGFGFIEVEG | X | X | X | IP |
| | (SEQ ID NO: 40) | | | | |
| CS 35(2, 3) | MLEGKVKWFNSEKGFGFIEVEGQDDVF | X | X | X | IP |
| | VHFSAIQG | | | | |
| | (SEQ ID NO: 41) | | | | |
| Ca Silk (4, 5) | SYGGEGIGNVAVAGELPVAGKTAVAGR | X | X | X | IP |
| | VPIIGAVGFGGPAGAAGAVSIAGR | | | | |
| | (SEQ ID NO: 42) | | | | |
| Cb Silk (4, 5) | GNLPFLGTAGVAGEFPTA | X | X | X | IP |
| | (SEQ ID NO: 43) | | | | |
| ccβ 6 | SIRELEARIRELELRIG | X | X | IP | IP |
| | (SEQ ID NO: 44) | | | | |
| E7^{A} | RAHYNIVTF | X | X | X | |
| | (SEQ ID NO: 45) | | | | |
| Sup35-6 (7) | GNNQQNY | X | X | IP | IP |
| | (SEQ ID NO: 46) | | | | |
| Sup35-7 (7) | NNQQNY | X | X | IP | IP |
| | (SEQ ID NO: 47) | | | | |
| BOC (8) | Boc-γ-aminobutyric acid-meta aminobenzoic | X | X | IP | IP |
| | acid-OMe | | | | |

^{A}E7 is a polypeptide from an HPV E7 polypeptide found to form amyloid-like aggregates.
IS = insoluble aggregate formed, CR = binds Congo red and shows polarized apple green birefringence, TT = shows enhanced fluorescence following Thioflavin T binding; EM = amyloid like fibrils by EM. X = positive result, IP = in progress
1 = Papanikolopoulou et al., J. Biol. Chem., 280:2481-2490 (2005).
2 = Wilkins et al., Eur. J. Biochem., 267 :2609-2616 (2000).
3 = Gross et al., Protein Sci., 8:1350-1357 (1999).
4 = Iconomidou et al., FEBS Lett., 499:268-273 (2001).
5 = Hamodrakas et al., J. Struct. Biol., 145 :226-235 (2004).
6 = Kammerer et al., Proc. Natl. Acad. Sci. USA, 101:4435-4440 (2004).
7 = Nelson et al., Nature, 435:773-778 (2005).
8 = Dutt et al., Org. Biomol. Chem., 3:2250-2254 (2005).

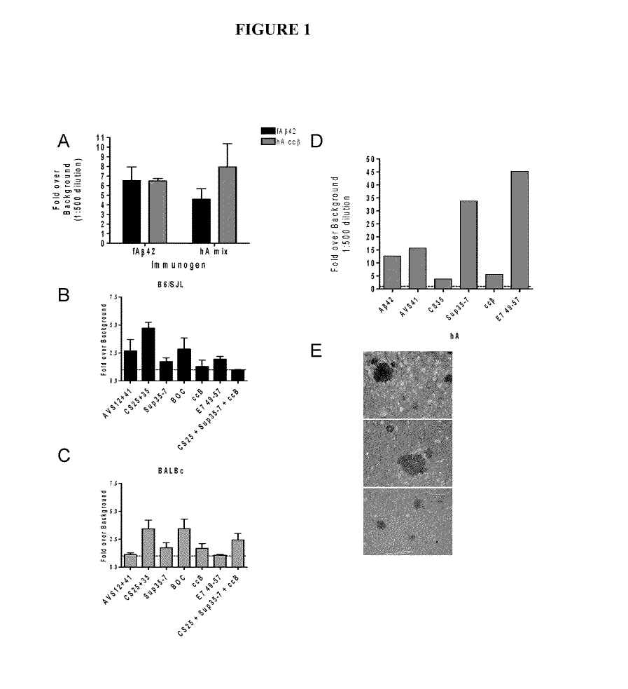

To determine if a generic anti-amyloid response is induced, reactivity of sera from fAβ42 immunized mice was analyzed against a panel of hAs. At 1:500 dilution, there was a significant IgM anti-amyloid titer against multiple hAs. Results are shown for reactivity of day 7 sera against ccβ amyloid and fAβ42 (FIG. 1A). These results are typical of the immune response seen at both 7 days and 30 days post immunization with fAβ42. A modest IgM anti-amyloid titer was induced. These results suggest that fAβ42 vaccines may be effective because they induce a “generic” anti-amyloid response. Encouraged by this result, hA vaccines were tested using a similar dosing paradigm to the fAβ42 vaccines described elsewhere (Schenk et al., Nature, 400:173-177 (1999) and Klein et al., Neurobiol. Aging, 25:569-580 (2004)). An example of the data produced by hA vaccination is provided (FIG. 1A). A mixture of hAs (hA mix; 1:1; CSP1-25:Sup35-7) induced an anti-amyloid titer of similar magnitude to fAβ42. The immune sera from the hA mix vaccinated mice recognized both fAβ42 and ccβ (FIG. 1A) as well as other hA.

A number of studies of TI2 antigens suggested that the response to a given TI2 antigen is variable in different mouse strains. This was found to be the case with hA antigens (FIGS. 1B and 1C). The anti-amyloid response to a given hA vaccine was variable depending on the strain of mouse used. For example, a response to hA from hA AVS was seen in B6/SJL mice, but not in BALBc. In contrast, both strains responded with an anti-amyloid response to CS25+35 (a 1:1 mixture of hAs from the cold shock protein polypeptides CS25 and CS35) and hA BOC formed from the dipeptide (Boc-γAbu-mABA-Ome). Indeed, such studies that involve surveying hA antigens revealed that both the mix of CS protein hAs and BOC produced the most reliable and highest titer anti-amyloid responses. For example, immunization with hA BOC produced a robust IgM titer on day 7 (FIG. 1D) and day 30 that cross reacts with fAβ42 and multiple other hAs.

To confirm that the antisera from hA immunized mice recognize Aβ amyloid, sections from 15 month old APP Tg2576 mice were stained. Antisera from hA immunized mice stained plaques at 1:100 and 1:500 dilutions (FIG. 1E). Consistent with ELISA data, only an IgM anti-amyloid response was induced by hA vaccination. Previous data reporting an IgG response was attributed to a cross-reactivity secondary.

Current results suggest that the following two hA vaccines are promising: a mixture of CS25 and CS35 polypeptides and hA derived from BOC. hA BOC produced a fairly robust anti-amyloid IgM response in 7 days. Such data strongly supports the assertion that amyloid is a TI2 antigen. The dose of immunogen and IL-12 may significantly impact the magnitude of the response. In addition, a first set of data from TcR αβ knockout mice revealed an equivalent response to hA BOC in the knockout mice as in the wild-type background.

### Example 2

### Isolation of Multiple Pan-Amyloid scFv Antibodies from a Phage Display Library

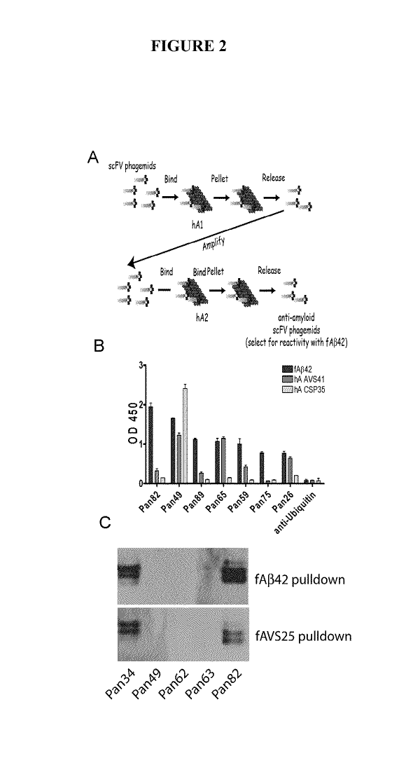

scFV expressing phagemid were prepared from the Tomlinson I and J libraries (MRC Centre for Protein Engineering, Cambridge, UK; world wide web at “geneservice.co.uk/products/proteomic/datasheets/tomlinsonIJ.pdf”). These libraries were based on a single human framework for VH and Vκ with side chain diversity incorporated at positions in the antigen-biding site that contact antigen. The two libraries have over 100 million different scFv fragments cloned in phagemid vectors. scFv fragments have a single polypeptide with the VH and VL domains attached to one another by a flexible glycine-serine linker To isolate putative anti-amyloid scFv, a modified panning protocol was used. Instead of binding the phagemid to amyloid on a solid surface, panning was performed by adding 200 μg of amyloid to ˜3-5 e¹¹ phagemid in solution, incubating the phagemid with the amyloid, and spinning down phagemid bound to the amyloid. Following washing of the amyloid pellet, phagemids were released by tryptic digest (FIG. 2A).

To isolate anti-amyloid scFvs, several different pans were conducted. In an initial panning experiment, several anti-amyloid scFvs were isolated. These scFvs were isolated by panning sequentially against: fAβ42, hA AVS 41, fAβ42, and then either CS35 or AVS41. Individual phagemid from the third and fourth rounds of panning were analyzed by ELISA for reactivity against fAβ42 fibrils. 15 clones with the highest reactivity to fAβ42 as determined by this ELISA were chosen for further analysis and sequencing. Based on highest relative reactivity to fAβ42 and hAs formed from AVS41 and CSP35, seven unique clones were selected and subcloned into plasmids suitable for eukaryotic expression. The reactivity of these scFv phagemid against multiple amyloids is shown in FIG. 2B. At least one scFv exhibited very high reactivity with fAβ42, and hAs from AVS41 and CS35, and multiple scFvs exhibited strong reactivity to fAβ42 and hA AVS41. In a second pan, the decision was to pan against multiple hAs and then test the phage from the final pan against fAβ42. In this case, the phage were panned against the following hAs in order: Sup35-6, AVS12, and CS25. This pan yielded three additional scFvs with high reactivity against fAβ42 that also bound to multiple hAs.

Almost all of the anti-amyloid scFv clones had amber stop codon mutants in the coding sequence. The amber codons code for glutamine when expressed in the TG-1 bacteria strain used to produce the phage, but can be repaired in order to express the scFvs in mammalian cells. Several of the anti-amyloid scFvs were repaired, expressed in mammalian cells, and shown to recognize fAβ42 and other hAs using anti-amyloid pulldown assays (FIG. 2C).

### Example 3

### Anti-Amyloid scFvs Attenuated Aβ Deposition

A recombinant antibody approach that uses rAAV vectors of serotype 1 to deliver anti-amyloid scFv directly into the brains of APP mice was used (Passini et al., J. Virol., 77:7034-7040 (2003)). Multiple anti-amyloid scFvs were isolated and shown to reduce amyloid burden in APP mice when delivered using a rAAV1.

AAV vectors containing the heavy and light chain constant regions linked by the 2A peptide were generated with restriction sites that enable rapid in frame insertion of the heavy and light chain variable regions (Fang et al., Nat. Biotechnol., 23:584-590 (2005)). These cassettes allow for the rapid cloning of the heavy and light chain variable regions from the anti-Aβ and anti-amyloid scFvs. Such vectors are capable of expressing high-levels of anti-amyloid and anti-Aβ intact immunoglobulins in situ following AAV mediated gene delivery.

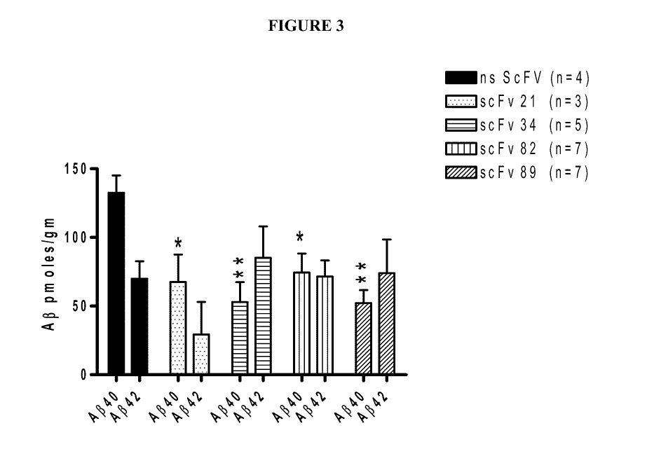

Newborn CRND8 mice were injected ICV with AAV1 expressing scFv 21, 34, 82, or 89. The anti-amyloid scFvs attenuated Aβ deposition in 3 month old CRND8 mice (FIG. 3).

### Example 4

### Isolation of scFv 4281, scFv 4281-6, scFv 55-1, and scFv 88-1 Antibodies from a Phage Display Library

scFv antibodies were isolated using methods similar to those described in Example 2. scFv 4281, scFv 4281-6, scFv 55-1, and scFv 88-1 antibodies were isolated by in vitro ribosome display panning sequentially against: fAβ42 and then lysozyme fibrils. The sequence of the lysozyme Gallus gallus fibrils is MRSLLILVLCFLPLAALGKVFGRCELAAAMKRHGLDNYRGYSLGNWVCVAKFESNFNTQATNRNTDGST DYGILQINSRWWCNDGRTPGSRNLCNIPCSALLSSDITASVNCAKKIVSDGNGMS AWVAWRNRCKGTDVQAWIRGCRL (SEQ ID NO:93). The isolated scFvs exhibited high reactivity against fAβ42 and also bound to multiple hAs (Table 2).

TABLE 2
Binding results.

OD Values

| Sample | Aβ fibrils | Lysozyme fibrils |
|---|---|---|
| scFv21 | 0.011 | 0.095 |
| scFv9 | 0.572 | 0.193 |
| scFv4281 | 0.083 | 0.261 |
| scFv55-1 | 0.255 | 0.445 |
| scFv4281-6 | 0.28 | 0.211 |
| scFv88-1 | 0.185 | 0.201 |

The anti-amyloid scFv 4281, scFv 4281-6, scFv 55-1, and scFv 88-1 clones had amber stop codon mutants in the coding sequence. The amber codons code for glutamine when expressed in the TG-1 bacteria strain used to produce the phage, but can be repaired in order to express the scFvs in mammalian cells. scFv 4281, scFv 4281-6, scFv 55-1, and scFv 88-1 were repaired, expressed in mammalian cells, and shown to recognize fAβ42 and hAs using an ELISA assay against lysozyme amyloid.

### Example 5

### scFv 4281, scFv 4281-6, scFv 55-1, and scFv 88-1 Antibodies Attenuated Aβ Deposition

A recombinant antibody approach that uses rAAV vectors of serotype 1 to deliver anti-amyloid scFv directly into the brains of APP mice was used (Passini et al., J. Virol., 77:7034-7040 (2003)). scFv 4281, scFv 4281-6, scFv 55-1, and scFv 88-1 antibodies reduced amyloid burden in APP mice when delivered using a rAAV1.

AAV vectors containing the heavy and light chain constant regions linked by the 2A peptide were generated with restriction sites that enable rapid in frame insertion of the heavy and light chain variable regions (Fang et al., Nat. Biotechnol., 23:584-590 (2005)). These cassettes allow for the rapid cloning of the heavy and light chain variable regions from the anti-Aβ and anti-amyloid scFvs (scFv 4281, scFv 4281-6, scFv 55-1, and scFv 88-1 antibodies). Such vectors are capable of expressing high-levels of anti-amyloid and anti-Aβ intact immunoglobulins in situ following AAV mediated gene delivery.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A substantially pure antibody having binding affinity for human amyloid and a heterologous amyloid.

2. The antibody of claim 1, wherein said antibody is a single chain variable fragment.

3. The antibody of claim 2, wherein said antibody has less than 10⁴ mol⁻¹ binding affinity for monomeric Aβ42.

4. The antibody of claim 2, wherein said antibody has less than two percent cross reactivity with monomeric Aβ42.

5. The antibody of claim 2, wherein said antibody is monoclonal.

6. The antibody of claim 2, wherein said human amyloid comprises human fibrillar Aβ42.

7. The antibody of claim 2, wherein said heterologous amyloid comprises Sup35-6.

8. A method for inhibiting Aβ plaque formation in a mammal, said method comprising administering an antibody to said mammal, wherein said antibody has binding affinity for human amyloid and a heterologous amyloid.

9. The method of claim 8, wherein said antibody is a single chain variable fragment.

10. The method of claim 9, wherein said antibody has less than 10⁴ mol⁻¹ binding affinity for monomeric Aβ42.

11. The method of claim 9, wherein said antibody has less than two percent cross reactivity with monomeric Aβ42.

12. The method of claim 9, wherein said antibody is monoclonal.

13. The method of claim 9, wherein said human amyloid comprises human fibrillar Aβ42.

14. The method of claim 9, wherein said heterologous amyloid comprises Sup35-6.
